(19) United States
(12) Patent Application Publication
(10) Pub. No.: US 2024/0026294 A1
(43) Pub. Date: Jan. 25, 2024

(54) NANOPARTICLE-MEDIATED IMMUNE CELL MANUFACTURE AND USE THEREOF

(71) Applicants: THE GEORGE WASHINGTON UNIVERSITY, Washington, DC (US); CHILDREN'S NATIONAL MEDICAL CENTER, Washington, DC (US)

(72) Inventors: ELIZABETH SWEENEY, McLean, VA (US); ROHAN FERNANDES, Bethesda, MD (US); MATTHEW TOVAR, Suffolk, VA (US); PALAK SEKHRI, Chevy Chase, MD (US); PREETHI BALA BALAKRISHNAN, Sunnyvale, CA (US); CONRAD RUSSELL CRUZ, Bethesda, MD (US)

(21) Appl. No.: 18/219,930

(22) Filed: Jul. 10, 2023

Related U.S. Application Data
(63) Continuation of application No. PCT/US22/11976, filed on Jan. 11, 2022.
(60) Provisional application No. 63/136,133, filed on Jan. 11, 2021.

(51) Int. Cl. C12N 5/0783; A61K 45/06; A61K 35/17

(52) U.S. Cl. CPC C12N 5/0638; A61K 45/06; A61K 35/17; C12N 2501/2306; C12N 2501/2307; C12N 2501/2312; C12N 2501/2315; C12N 2501/22; C12N 2501/25; C12N 2501/2301; C12N 2501/2304

(57) ABSTRACT

The present disclosure generally relates target cancer cell specific immunotherapy compositions, methods of making and use thereof. Also provided in the disclosure are methods of treating a cancer in a subject in need thereof.

### CROSS REFERENCE TO RELATED APPLICATION

This application is a continuation of PCT App. No. PCT/US22/11976, tiled Jan. 11, 2022, for NANOPARTICLE-MEDIATED IMMUNE CELL MANUFACTURE AND USES THEREOF, which claims the benefit of U.S. Provisional Application No. 63/136,133, tiled on Jan. 11, 2021, the disclosures of which are hereby incorporated by reference in their entireties.

### FIELD

Embodiments of the instant disclosure relate to novel compositions and methods for generating immunotherapy compositions. In some embodiments, immunotherapy compositions disclosed herein can target a specific cancer cell. Also provided in the instant disclosure are methods of treating a cancer in a subject in need thereof.

### BACKGROUND

Cell-based immunotherapies have shown to be effective for treating a variety of immune-related diseases, including cancers. However, due to complex interactions and reactions of endogenous immune system to tumors, it is challenging to produce immunotherapies through conventional approaches. Since there are multiple events of immune dysfunction, immune escape, and immune suppression in a cancer patient's body, the tumor is sheltered from immune attack. In particular, immune cells are prevented from trafficking to the tumor. Due to the immune dysfunction, the patient's body can become tolerant of the tumor's presence. Immunotherapies based on immune cells, such as T cells, may be promising treatments for cancer patients; however, there are unmet needs of generating multi antigen-specific immune cells which have specificities to many endogenous antigens, such as superior specificity of T cells to tumor cells. There are also needs in the field to increase immune cell expansion, such as T cell expansion, to enable a faster and cheaper manufacture timeline.

### SUMMARY

Embodiments of the instant disclosure relate to novel compositions and methods for generating immunotherapy compositions. Certain embodiments of the present disclosure provide immunotherapy compositions specific to at least one target cancer cell. In certain embodiments, immunotherapy compositions disclosed herein may comprise ex vivo expanded immune cells, wherein the ex vivo expanded immune cells may be specific to at least one target cancer cell by co-culturing immune cells during ex vivo expansion with at least one target cancer cell that has been exposed to at least one Prussian blue nanoparticle (PBNP).

In some embodiments, PBNPs herein may comprise a Prussian blue material represented by general formula (I):

AₓB_{y}M₄[M′(CN)₆]_{z}·nH₂O (I)

wherein: A represents at least one of VO₂, Ca, V, Cr, Mn, Fe, Co, Ni, Cu, Zn, Ga, Sr, Zr, Nb, Li, Na, K, Rb, Cs, Fr, Tl, Mo, Ru, Rh, Pd, Ag, Cd, In, Lu, Ba, Hf, Ta, W, Os, Pt, Hg, La, Eu, Gd, Tb, Dy and Ho, in any oxidation state and any combination thereof; B represents at least one of VO₂, Ca, V, Cr, Mn, Fe, Co, Ni, Cu, Zn, Ga, Sr, Zr, Nb, Li, Na, K, Rb, Cs, Fr, Tl, Mo, Ru, Rh, Pd, Ag, Cd, In, Lu, Ba, Hf, Ta, W, Os, Pt, Hg, La, Eu, Gd, Tb, Dy and Ho, in any oxidation state and any combination thereof; M represents at least one of VO₂, Ca, V, Cr, Mn, Fe, Co, Ni, Cu, Zn, Ga, Sr, Zr, Nb, Li, Na, K, Rh, Cs, Fr, Tl, Mo, Ru, Rh, Pd, Ag, Cd, In, Lu, Ba, Hf, Ta, W, Os, Pt, Hg, La, Eu, Gd, Tb, Dy and Ho, in any oxidation state and any combination thereof; M′ represents at least one of VO₂, Ca, V, Cr, Mn, Fe, Co, Ni, Cu, Zn, Ga, Sr, Zr, Nb, Li, Na, K, Rb, Cs, Fr, Tl, Mo, Ru, Rh, Pd, Ag, Cd, In, Lu, Ba, Hf, Ta, W, Os, Pt, Hg, La, Eu, Gd, Tb, Dy and Ho, in any oxidation state and any combination thereof; x is from 0.1 to about 1; y is from 0.1 to about 1; z is from 0.1 to about 4; and n is from 0.1 to about 24.

In some embodiments, immunotherapy compositions disclosed herein may comprise at least one target cancer cell exposed to at least one PBNP wherein both have been subjected to photothermal therapy. In accordance with these embodiments, photothermal therapy may comprise use of a device that emits electromagnetic radiation with a wavelength that irradiates the at least one PBNP in the presence of the at least one target cancer cell. In some aspects, a wavelength that irradiates the at least one PBNP in the presence of the at least one target cancer cell may be about 300 nm to about 1200 nm.

In some embodiments, immunotherapy compositions disclosed herein may comprise immune cells isolated from a mammal. In some embodiments, immunotherapy compositions disclosed herein may comprise immune cells isolated from a human. In some embodiments, immunotherapy compositions disclosed herein may comprise immune cells that are allogeneic, autologous, or a combination thereof. In some aspects, immunotherapy compositions disclosed herein may comprise immune cells that are autologous.

In some embodiments, immunotherapy compositions disclosed herein may comprise immune cells obtained from peripheral blood mononuclear cells (PBMCs), leukapheresis sample, tumor-infiltrated lymphocytes, tissue-infiltrated lymphocytes, lymph nodes, thymus, secondary lymphoid organs, or any combination thereof.

In some embodiments, immunotherapy compositions disclosed herein may comprise target cancer cells isolated from a mammal having or suspected of having a cancer. In some embodiments, immunotherapy compositions disclosed herein may comprise target cancer cells isolated from a human having or suspected of having a cancer. In some embodiments, immunotherapy compositions disclosed herein may comprise target cancer cells that are allogeneic, autologous, or a combination thereof. In some aspects, immunotherapy compositions disclosed herein may comprise target cancer cells that are autologous.

In some embodiments, immunotherapy compositions disclosed herein may comprise ex vivo expanded immune cells comprised of T cells. In accordance with these embodiments, ex vivo expanded immune cells disclosed herein may comprise CD8+ T cells, CD4+ T cells, or any combination thereof.

Certain embodiments of the present disclosure provides methods of preparing an immunotherapy composition as disclosed herein. In some embodiments, methods disclosed herein can be used to prepare an immunotherapy composition specific to at least one target cancer. In accordance with these embodiments, methods of preparing an immunotherapy composition disclosed herein may comprise: a) obtaining an initial immune cell population; b) isolating monocyte-derived dendritic cells (DCs) and T cells from the initial immune cell population; c) obtaining at least one target cancer cell; d) exposing the at least one target cancer cell to at least one Prussian blue nanoparticle (PBNP) and subjecting the at least one target cancer cell and the at least one PBNP to photothermal therapy (PTT); e) co-culturing the isolated monocyte-derived DCs with the at least one target cancer cell subjected to PTT; f) expanding the co-culture in a medium comprising the isolated T cells; and g) harvesting T cells specific to the at least one target cancer cell from the expanded co-culture. In some embodiments, methods herein of co-culturing of isolated monocyte-derived DCs with at least one target cancer cell subjected to PTT may further comprise co-culturing in the presence of GM-CSF, TNF-a, IL-1b, IL4, IL-6, GM-CSF, IFN-γ, IL-4, lipopolysaccharide, or any combination thereof.

In some embodiments, methods herein may further comprise stimulating isolated T cells with DCs harvested from the co-culture. In some embodiments, methods herein may further comprise stimulating isolated T cells with DCs harvested from the co-culture at an about 1:10 (DC:T cell) ratio, an about 1:9 (DC:T cell) ratio, an about 1:8 (DC:T cell) ratio, an about 1:7 (DC:T cell) ratio, an about 1:6 (DC:T cell) ratio, an about 1:5 (DC:T cell) ratio, an about 1:3 (DC:T cell) ratio, an about 1:2 (DC:T cell) ratio, or an about 1:1 (DC:T cell) ratio. In some embodiments, methods herein may further comprise stimulating isolated T cells with DCs harvested from the co-culture in the presence of IL-6, IL-7, IL-12, IL-15, or any combination thereof.

In some embodiments, methods of preparing an immunotherapy composition disclosed herein may assess T cells specific to at least one target cancer cell harvested from the expanded co-culture for the presence of at least one markers of T cell activation. In accordance with these embodiments, markers of T cell activation may comprise CD45RO, CD137, CD25, CD279, CD179, CD62L, HLA-DR, CD69, CD223 (LAG3), CD134 (0X40), CD183 (CXCR3), CD27 (IL-7Ra), CD366 (TIM3), CD80, CD152 (CTLA-4), CD28, CD278 (ICOS), CD154(CD40L), or any combination thereof. In some aspects, methods of preparing an immunotherapy composition disclosed herein may assess T cells specific to at least one target cancer cell harvested from the expanded co-culture for the presence of at least one markers of T cell activation, wherein at least one marker of T cell activation is expressed at an amount that is higher than a native T cell. In some aspects, methods of preparing an immunotherapy composition disclosed herein may assess T cells specific to at least one target cancer cell harvested from the expanded co-culture for the presence of at least one markers of T cell activation, wherein at least one marker of T cell activation is expressed at an amount that is at least about 1% to at least about 90% higher (e.g., at least about 1%, about 5%, about 10%, about 20%, about 30%, about 40%, about 50%, about 60%, about 70%, about 80%, about 90% higher) than a native T cell.

In some embodiments, methods of preparing an immunotherapy composition disclosed herein may include obtaining target cancer cells from a breast cancer, colorectal cancer, head and neck cancer, kidney cancer, lung cancer, ovarian cancer, pancreatic cancer, prostate cancer, brain cancer, adenoid cystic carcinoma, anaplastic astrocytoma, anaplastic ependymoma, anaplastic oligodendroglioma, brainstem glioma, diffuse astrocytoma, diffuse intrinsic pontine glioma (DIPG), ganglioglioma, medulloblastoma, pilocytic astrocytoma, cholangiocarcinoma, chronic atypical myelogenous leukemia, endometrial carcinoma, esophageal cancer, Ewing sarcoma, gastrointestinal stromal tumor (GIST), leptomeningeal carcinomatosis, multiple myeloma, myelodysplastic syndrome, neuroendocrine carcinoma, Non-Hodgkin's lymphoma, pleomorphic sarcoma, Primitive neuroectodermal tumor (PNET), refractory anemia, salivary gland carcinoma, skin cancer, stomach cancer, thyroid cancer, urothelial cancer, or any combination thereof.

In some embodiments, methods of preparing an immunotherapy composition disclosed herein may include obtaining an initial immune cell population that can be matched on at least 1 HLA to the at least one target cancer cell.

Certain embodiments of the present disclosure provide methods for treating a subject with an immunotherapy composition disclosed herein. In some embodiments, methods of treating a subject in need thereof may comprise: a) isolating initial immune cell population from the subject; b) isolating monocyte-derived dendritic cells (DCs) and T cells from the initial immune cell population; c) obtaining at least one target cancer cell from the subject; d) exposing the at least one target cancer cell to at least one Prussian blue nanoparticle (PBNP) and subjecting the at least one target cancer cell and the at least one PBNP to photothermal therapy (PTT); e) co-culturing the isolated monocyte-derived DCs with the at least one target cancer cell subjected to PTT; f) expanding the co-culture in a medium comprising the isolated T cells; g) harvesting T cells specific to at least one target cancer cell from the expanded co-culture; h) administering to the subject in need thereof, an effective amount of the ex vivo expanded T cells specific to at the least one target cancer cell from step h.

In some embodiments, a subject in need thereof can be human patient diagnosed as having, or is suspected of having a cancer. In accordance with these embodiments, a cancer to be treated by methods disclosed herein may be a solid tumor and/or metastatic cancer. In some embodiments, a cancer to be treated by methods disclosed herein may be breast cancer, colorectal cancer, head and neck cancer, kidney cancer, lung cancer, ovarian cancer, pancreatic cancer, prostate cancer, brain cancer, adenoid cystic carcinoma, anaplastic astrocytoma, anaplastic ependymoma, anaplastic oligodendroglioma, brainstem glioma, diffuse astrocytoma, diffuse intrinsic pontine glioma (DIPG), ganglioglioma, medulloblastoma, pilocytic astrocytoma, cholangiocarcinoma, chronic atypical myelogenous leukemia, endometrial carcinoma, esophageal cancer, Ewing sarcoma, gastrointestinal stromal tumor (GIST), leptomeningeal carcinomatosis, multiple myeloma, myelodysplastic syndrome, neuroendocrine carcinoma, Non-Hodgkin's lymphoma, pleomorphic sarcoma, Primitive neuroectodermal tumor (PNET), refractory anemia, salivary gland carcinoma, skin cancer, stomach cancer, thyroid cancer, urothelial cancer, or any combination thereof.

In some embodiments, methods of treating a subject in need thereof may comprise administration of the ex vivo expanded T cells specific to at least one target cancer cell to the subject by infusion. In some embodiments, methods of treating a subject in need thereof may further comprise administration of one or more therapeutic regimens, wherein the one or more therapeutic regimens may comprise administration of a chemotherapeutic agent, administration of a monoclonal antibody therapy, administration of a small molecule, or any combination thereof. In some aspects, administration of one or more monoclonal antibody therapies in combination with the methods herein may be selected from the group consisting of adotrastuzumab, trastuzumab, pertuzumab, cetuximab, panitumumab, necitumumab, ramucirumab, bevacizumab, rituximab, ofatumumab, ibritumomab, tositumomab, obinutuzumab, inotuzumab, alemtuzumab, gemtuzumab, brentuximab, blinatumomab, daratumumab, ipilimumab, nivolumab, atezolizumab, avelumab, cemiplimab, pembrolizumab, durvalumab, denosumab, dinutuximab, olaratumab, and elotuzumab. In some aspects, administration of one or more small molecules in combination with the methods herein may be selected from the group consisting of imatinib, dasatinib, nilotinib, bosutinib, regorafenib, ponatinib, sunitinib, sorafenib, erdafitinib, lenvatinib, pazopanib, afatinib, gefitinib, osimertinib, vandetanib, erlotinib, lapatinib, dacomitinib, neratinib, ribociclib, abemaciclib, palbociclib, cabozantinib, crizotinib, axitinib, alectinib, vemurafenib, encorafenib, dabrafenib, olaparib, rucaparib, talazoparib, niraparib, larotrectinib, entrectinib, lorlatinib, ibrutinib, cobimetinib, binimetinib, trametinib, brigatinib, cgilteritinib, ceritinib, ivosidenib, carfilzomib, marizomib, alpelisib, duvelisib, and copanlisib.

In certain embodiments, pharmaceutical compositions comprising any one or more of the immunotherapy compositions disclosed herein and a pharmaceutically acceptable excipient are provided herein. In some embodiments, pharmaceutical compositions disclosed herein may further comprise one or more additional agents. In accordance with these embodiments, the additional agents may comprise one or more anti-cancer agents.

In certain embodiments, kits for ex vivo immune cell expansion of T cells specific to at least one target cancer cell are provided herein. In some embodiments, kits disclosed herein can comprise at least one culture medium and at least one Prussian blue nanoparticle (PBNP). In some aspects, kits disclosed herein can further comprise GM-CSF, TNF-a, IL-1b, IL4, IL-6, GM-CSF, IFN-γ, IL-4, lipopolysaccharide, IL-7, IL-12, IL-15, or any combination thereof.

### BRIEF DESCRIPTION OF THE FIGURES

The patent or application file contains at least one drawing executed in color. Copies of this patent or patent application with color drawing(s) will be provided by the Office by request and payment of the necessary fee

The following drawings form part of the present specification and are included to further demonstrate certain embodiments of the present disclosure. Certain embodiments can be better understood by reference to one or more of these drawings in combination with the detailed description of specific embodiments presented herein.

FIG. 1 depicts a schematic showing a treatment scheme according to an exemplary embodiment.

FIG. 2 depicts a schematic showing a first manufacture protocol according to an exemplary embodiment.

FIG. 3 depicts a schematic showing a second manufacture protocol according to an exemplary embodiment.

FIGS. 4A-4B depict graphs showing PBNP-PTT heated U87 (GBM) cells in laser power-dependent manner (FIG. 4A) and at a thermal dose (FIG. 4B).

FIGS. 5A-5B depict graphs showing PBNP-PTT heated SNB-19 (GBM) cells in laser power-dependent manner (FIG. 5A) and at a thermal dose (FIG. 5B).

FIGS. 6A-6B depict graphs showing PBNP-PTT heated SH-SY5Y (neuroblastoma) cells in laser power-dependent manner (FIG. 6A) and at a thermal dose (FIG. 6B).

FIGS. 7A-7B depict graphs showing PBNP-PTT heated MDA-MB-231 (breast cancer) cells in laser power-dependent manner (FIG. 7A) and at a thermal dose (FIG. 7B).

FIG. 8 depicts a graph showing tumor cell lines PBNP-PTT heated to a thermal dose ˜10 log (CEM43).

FIGS. 9A-9C depict graphs showing PBNP-PTT increased immunogenicity of U87 cells as shown by ATP expression (FIG. 9A), HMGB1 expression (FIG. 9B), and calreticulin expression (FIG. 9C).

FIGS. 10A-10C depict graphs showing PBNP-PTT increased immunogenicity of SNB-19 cells as shown by cell viability (FIG. 10A), ATP release (FIG. 10B), and calreticulin expression (FIG. 10C).

FIGS. 11A-11C depict graphs showing PBNP-PTT increased immunogenicity of SH-SY5Y cells as shown by cell viability (FIG. 11A), ATP release (FIG. 11B), and calreticulin expression (FIG. 11C).

FIGS. 12A-12C depict graphs showing PBNP-PTT increased immunogenicity of MDA-MB-231 cells as shown by cell viability (FIG. 12A), ATP release (FIG. 12B), and calreticulin expression (FIG. 12C).

FIG. 13 depicts a schematic showing a T cell manufacture protocol via PBNP-PTT (i.e., PBNP-PTT-mediated expansion) according to an exemplary embodiment.

FIG. 14 depicts a schematic showing a T cell manufacture protocol via freeze-thaw (i.e., lysate-mediated expansion) according to an exemplary embodiment.

FIGS. 15A and 15B depicts representative images showing T cells expanded from U87 cells not subjected to PBNP-PTT (FIG. 15A) and U87 cells that were subjected to PBNT-PTT (FIG. 15B) at day 11 for 7 days post-Stim no. 1.

FIGS. 16A and 16B depict graphs showing manufacture of U87-specific T cells, which was enabled using PBNP-PTT.

FIGS. 17A-17C depict graphs showing the manufacture of U87-specific T cells, which was enabled using PBNP-PTT, regarding post-Stim 1 (FIG. 17A), post-Stim 2 (FIG. 17B), and post-Stim 3 (FIG. 17C).

FIGS. 18A and 18B depict graphs showing PBNP-PTT-mediated development expanded T cells in SNB19 cells (FIG. 18A) and U87 cells (FIG. 18B).

FIGS. 19A and 19B depict graphs showing PBNP-PTT-mediated development expands T cells when using the cancer cell lines SNB19, U87, SH-SY5Y, MDA-MB-231, HCC1599BL, and MDA-MB -231+HCC1599BL.

FIG. 20 depicts a graph showing the generation of U87-specific T cells in the absence of cytokines, which was enabled using PBNP-PTT.

FIG. 21 depicts representative images of flow cytometry plots showing that PBNP-PTT-mediated cell expansion resulted in the manufacture of primarily CD8+ U87-specific T cells.

FIGS. 22A and 22B depict graphs showing T cell expansion using a lysate-mediated method in cancer cell lines.

FIG. 23 depicts a graph showing that a majority of cells expanded via PBNP-PTT consisted of a T cell population.

FIGS. 24A and 24B depict graphs showing mixed CD4+ and CD8+ T cell populations within the T cells expanded via PBNP-PTT.

FIGS. 25A and 25B depict graphs showing high CD4+ T cell populations within the T cells expanded via the lysate-mediated expansion.

FIGS. 26A-26C depict representative images showing ELISPOT plates developed for U87-specific T cells post stimulation #1 (FIG. 26A), #2 (FIG. 26B), and #3 (FIG. 26C).

FIGS. 27A-27C depict graphs showing IFN-γ secretion in response to target cells from T cells developed via PBNP-PTT-mediated expansion.

FIG. 28 depicts a graph showing that T cells developed via PBNP-PTT-mediated expansion secreted IFN-65 in response to target cells via MHC-I signaling.

FIGS. 29A and 29B depict graphs showing IFN-65 secretion in response to target cells from T cells developed via lysate-mediated expansion.

FIGS. 30A-30D depict graphs showing cytotoxicity of T cells developed via PBNP-PTT-mediated expansion against target cells.

FIG. 31 depicts a graph showing cytotoxicity of T cells developed via lysis-mediated expansion against target cells.

FIG. 32 depicts a graph showing lack of cytotoxicity by T cells developed via PBNP-PTT-mediated expansion against unmatched normal human astrocytes (NHAs).

FIG. 33 depicts a graph showing lack of cytotoxicity by SH-SY5Y T cells developed via PBNP-PTT-mediated expansion using cells from a mismatched donor against target cells.

FIGS. 34A and 34B depict graphs showing cytotoxicity to off-target cells in T cells generated via PBNP-PTT-mediated expansion compared to those generated via lysate-mediated expansion.

FIG. 35 depicts a graph showing that T cells generated via PBNP-PTT were more cytotoxic toward U87 cells than tumor-associated antigens (TAA T) cells.

### DEFINITIONS

Terms, unless defined herein, have meanings as commonly understood by a person of ordinary skill in the art relevant to certain embodiments disclosed herein or as applicable.

As used herein “about” unless otherwise indicated, applies to all numbers expressing quantities of agents and/or compounds, properties such as molecular weights, reaction conditions, and as disclosed herein are contemplated as being modified in all instances by this term. Accordingly, unless indicated to the contrary, the numerical parameters in the specification and claims are approximations that can vary from about 10% to about 15% plus and/or minus depending upon the desired properties sought as disclosed herein. Numerical values as represented herein inherently contain standard deviations that necessarily result from the errors found in the numerical value's testing measurements.

As used herein, “individual”, “subject”, “host”, and “patient” can be used interchangeably herein and refer to any mammalian subject for whom diagnosis, treatment, prophylaxis or therapy is desired, for example, humans, pets, livestock, horses or other animals.

As used herein, “treat,” “treating,” or “treatment” can refer to reversing, ameliorating, or inhibiting onset or inhibiting progression of a health condition or disease or a symptom of the health condition or disease.

As used herein, “marker” can refer to any molecule that can be measured or detected, for example. For example, a marker can include, without limitations, a nucleic acid, such as, a transcript of a gene, a polypeptide product of a gene, a glycoprotein, a carbohydrate, a glycolipid, a lipid, a lipoprotein, a carbohydrate, and/or a small molecule. As used herein, “expression” and grammatical equivalents thereof, in the context of a marker, can refer to production of the marker as well as level or amount of the marker.

### DETAILED DESCRIPTION

In the following sections, certain exemplary compositions and methods are described in order to detail certain embodiments of the invention. It will be obvious to one skilled in the art that practicing the certain embodiments does not require the employment of all or even some of the specific details outlined herein, but rather that concentrations, times and other specific details can be modified through routine experimentation. In some cases, well known methods, or components have not been included in the description.

Embodiments of the present disclosure relate to novel compositions and methods for generating ex vivo expanded immune cells specific to at least one target cell. Some embodiments of the present disclosure relate to novel compositions and methods for generating ex vivo expanded immune cells specific to at least one target cancer/target cancer cell. In certain embodiments, ex vivo expanded immune cells generated as disclosed herein may be and/or may comprise an immunotherapy composition (e.g., a pharmaceutical composition comprising an immunotherapy composition disclosed herein). In certain embodiments, ex vivo expanded immune cells generated as disclosed herein may be a pharmaceutical composition. In certain embodiments, ex vivo expanded immune cells generated as disclosed herein may be used to treat, prevent, and/or attenuate a cancer.

### (I) Compositions

Aspects of the present disclosure include compositions encompassing at least one immune cell specific to at least one target cancer. Compositions disclosed herein may encompass at least one immune cell specific to at least one target cancer wherein cancer type specificity can be a result of any one of the methods disclosed herein.

### (a) Immune Cells

In some embodiments, compositions disclosed herein can include at least one immune cell. As used herein an “immune cell” refers to a cell of the immune system. Immune cells can be categorized as lymphocytes, neutrophils, granulocytes, mast cells, monocytes/macrophages, and dendritic cells. In some aspects, compositions disclosed herein can include at least one lymphocyte. In some aspects, lymphocytes can be T-cells (CD4+ T cells and/or CD8+ T cells), B-cells, and natural killer (NK) cells are categorized as lymphocytes. In other aspects, an immune cell disclosed herein can be cytotoxic lymphocyte. As used herein, a “cytotoxic lymphocyte” refers to a lymphocyte capable cytolysis. For example, but not limited to, a cytotoxic lymphocyte can be capable of killing cancer cells, cells that are infected (particularly with viruses), and cells that are damaged in one or more other ways. In some aspects, compositions disclosed herein can include at least one dendritic cell. A dendritic cell is a type of phagocyte and a type of antigen-presenting cell (APC). In some embodiments, dendritic cells may be tolerogenic dendritic cells (iDCs) and/or control dendritic cells (cDCs). In some embodiments, DCs may express a combination of markers, for example, CD11c+, CD45+; CD83+ HLA-DR+CD11c+; MHCII+, CD11c+, CD80+, CD40+, CD86+; CD1B+, CD5+, CD19+, IL10+; CD19+, CD27+, CD38, CD24+; or others. Memory DC populations may additionally express CD27+. Positive expression levels of cell markers may vary between experimental samples, vary between cell populations, vary between subjects from which they are isolated, vary within a subpopuiation, or combinations thereof. Expression of a marker may be determined by any methods known to those of skill in the art. In some non-limiting examples, expression can be determined by FACS sorting using a standard method for gating for high and low expressing cells. In some embodiments, DCs may be isolated from other immune cells (e.g., T cells) in a sample by CD14 selection.

In some embodiments, an immune cell can be isolated from a subject. In some aspects, an immune cell can be isolated from peripheral blood, umbilical cord blood, and/or bone marrow. In other aspects, an immune cell can be isolated from peripheral blood mononuclear cells (PBMCs). In still other aspects, an immune cell can be isolated from a leukapheresis sample. In yet other aspects, an immune cell can be isolated from tumor-infiltrated lymphocytes, tissue-infiltrated lymphocytes, lymph nodes, thymus, and/or secondary lymphoid organs.

In certain embodiments, an immune cell can be isolated from autologous peripheral blood, umbilical cord blood, bone marrow, PBMCs, leukapheresis sample, tumor-infiltrated lymphocytes, tissue-infiltrated lymphocytes, lymph nodes, thymus, and/or secondary lymphoid organs. As used herein, the term “autologous” refers to peripheral blood, umbilical cord blood, bone marrow, PBMCs, leukapheresis sample, tumor-infiltrated lymphocytes, tissue-infiltrated lymphocytes, lymph nodes, thymus, and/or secondary lymphoid organs obtained from the same subject to be treated with the compositions disclosed herein. In other embodiments, an immune cell can be isolated from allogeneic peripheral blood, umbilical cord blood, bone marrow, PBMCs, leukapheresis sample, tumor-infiltrated lymphocytes, tissue-infiltrated lymphocytes, lymph nodes, thymus, and/or secondary lymphoid organs. As used herein, the term “allogeneic” refers to peripheral blood, umbilical cord blood, bone marrow, PBMCs, leukapheresis sample, tumor-infiltrated lymphocytes, tissue-infiltrated lymphocytes, lymph nodes, thymus, and/or secondary lymphoid organs obtained from a different subject of the same species as the subject to be treated with the compositions disclosed herein. In some aspects, an immune cell can be isolated from haploidentical allogeneic peripheral blood, umbilical cord blood, bone marrow, PBMCs, leukapheresis sample, tumor-infiltrated lymphocytes, tissue-infiltrated lymphocytes, lymph nodes, thymus, and/or secondary lymphoid organs.

In some embodiments, at least one immune cell can be subjected to ex vivo expansion following isolation from peripheral blood, umbilical cord blood, bone marrow, PBMCs, leukapheresis sample, tumor-infiltrated lymphocytes, tissue-infiltrated lymphocytes, lymph nodes, thymus, and/or secondary lymphoid organs. In some aspects, immune cells at an amount of about 0.5×10⁶ cells/milliliter (ml) to about 1×10⁷ cells/ml, about 1×10⁶ cells/milliliter (ml) to about 9×10⁶ cells/ml, or about 2×10⁶ cells/milliliter (ml) to about 8×10⁶ cells/ml can be subjected to ex vivo expansion following isolation from a subject. In other aspects, immune cells at an amount of about 0.5×10⁶ cells/ml, about 1×10⁶ cells/ml, about 2×10⁶ cells/ml, about 3×10⁶ cells/ml, about 4×10⁶ cells/ml, about 5×10⁶ cells/ml, about 6×10⁶ cells/ml, about 7×10⁶ cells/ml, about 8×10⁶ cells/ml, about 9×10⁶ cells/ml, or about 1×10⁷ cells/ml can be subjected to ex vivo expansion following isolation from a subject. In some embodiments, at least one DC can be subjected to ex vivo expansion following isolation from peripheral blood, umbilical cord blood, bone marrow, PBMCs, leukapheresis sample, tumor-infiltrated lymphocytes, tissue-infiltrated lymphocytes, lymph nodes, thymus, and/or secondary lymphoid organs. In some embodiments, at least one DC can be subjected to ex vivo expansion following isolation from peripheral blood, umbilical cord blood, bone marrow, PBMCs, leukapheresis sample, tumor-infiltrated lymphocytes, tissue-infiltrated lymphocytes, lymph nodes, thymus, and/or secondary lymphoid organs by CD14 selection.

In some embodiments, an initial immune cell population can be prepared from peripheral blood, umbilical cord blood, bone marrow, PBMCs, leukapheresis sample, tumor-infiltrated lymphocytes, tissue-infiltrated lymphocytes, lymph nodes, thymus, and/or secondary lymphoid organs. In some embodiments, an initial cell population may be frozen and thawed at least, at least twice, or at least three times prior to use in the methods disclosed herein. In some embodiments, an initial immune cell population can isolated from peripheral blood, umbilical cord blood, bone marrow, PBMCs, leukapheresis sample, tumor-infiltrated lymphocytes, tissue-infiltrated lymphocytes, lymph nodes, thymus, and/or secondary lymphoid organs by phenotypic selection for one or more markers. Phenotypic selection for one or more markers can include, but not limited to CD3, CD28, CD3/CD28, or CD14 selection. In some embodiments, dendritic cells are isolated, as measured by CD11c expression on their surface. In some embodiments, T cells are isolated, as measured by CD3 expression on their surface.

### (b) Target Cells

In some embodiments, compositions disclosed herein can include at least one target cell. As used herein, a “target cell” refers to the cell to be the targeted by immune responses generated from the ex vivo immune cells generated as disclosed herein. In some embodiments, target cells may be obtained from surgery, biopsy, cell banks, commercial vendors, repositories, healthy donors, or a combination thereof. In some embodiments, a target cell is can be autologous and/or allogeneic. In preferred embodiments, a target cell is autologous.

In some embodiments, a target cell can be a cancer cell. In some embodiments, a target subject having or suspected of having a cancer. In some embodiments, a target cell can be at least a cell obtained from a breast cancer, colorectal cancer, head and neck cancer, kidney cancer, lung cancer, ovarian cancer, pancreatic cancer, prostate cancer, brain cancer, adenoid cystic carcinoma, anaplastic astrocytoma, anaplastic ependymoma, anaplastic oligodendroglioma, brainstem glioma, diffuse astrocytoma, diffuse intrinsic pontine glioma (DIPG), ganglioglioma, medulloblastoma, pilocytic astrocytoma, cholangiocarcinoma, chronic atypical myelogenous leukemia, endometrial carcinoma, esophageal cancer, Ewing sarcoma, gastrointestinal stromal tumor (GIST), leptomeningeal carcinomatosis, multiple myeloma, myelodysplastic syndrome, neuroendocrine carcinoma, Non-Hodgkin's lymphoma, pleomorphic sarcoma, Primitive neuroectodermal tumor (PNET), refractory anemia, salivary gland carcinoma, skin cancer, stomach cancer, thyroid cancer, urothelial cancer, or any combination thereof.

In some embodiments, target cells for use herein may be subjected to one or agents. In some embodiments, target cells for use herein may be subjected one or more compositions comprising PBNP (a PBNP composition) as disclosed herein. In some embodiments, target cells for use herein may be subjected to PBNP-PTT. In some embodiments, target cells for use herein may be lysed after PBNP-PTT. Non-limiting examples of methods of lysing target cells suitable for use herein can include mechanical disruption, liquid homogenization, high frequency sound waves, freeze/thaw cycles, manual grinding, or cultured in serum-rich or serum-free media for 1-120 hours post-PBNP-PTT.

### (c) Prussian Blue Materials

In certain embodiments, compositions disclosed herein comprise a nanoparticle formed of at least one or more Prussian blue materials. As used herein, “Prussian blue materials”, “Prussian blue” and “Prussian blue compounds” are used interchangeably. Unless indicated otherwise, the symbols used to represent the elements of which the Prussian blue materials and/or analogs thereof of the present disclosure are comprised are the symbols used in the periodic table of elements to represent the chemical elements (for example, “Fe” represents iron, etc.).

In certain embodiments, compositions disclosed herein may comprise doped Prussian blue compounds. In various aspects, compositions disclosed herein may comprise Prussian blue materials represented by general formula (I):

AₓB_{y}M₄[M′(CN)₆]_{z}·nH₂O (I),

which is coated with a biocompatible shell onto which targeting, imaging and/or therapeutic agents are attached. In the compounds of general formula (I),

A represents at least one of VO₂, Ca, V, Cr, Mn, Fe, Co, Ni, Cu, Zn, Ga, Sr, Zr, Nb, Li, Na, K, Rb, Cs, Fr, Tl, Mo, Ru, Rh, Pd, Ag, Cd, In, Lu, Ba, Hf, Ta, W, Os, Pt, La, Eu, Gd, Tb, Dy and Ho, in any oxidation state and in any combination thereof;

B represents at least one of VO₂, Ca, V, Cr, Mn, Fe, Co, Ni, Cu, Zn, Ga, Sr, Zr, Nb, Li, Na, K, Rb, Cs, Fr, Tl, Mo, Ru, Rh, Pd, Ag, Cd, In, Lu, Ba, Hf, Ta, W, Os, Pt, Hg, La, Eu, Gd, Tb, Dy and Ho, in any oxidation state and in any combination thereof;

M represents at least one of VO₂, Ca, V, Cr, Mn, Fe, Co, Ni, Cu, Zn, Ga, Sr, Zr, Nb, Li, Na, K, Rb, Cs, Fr, Tl, Mo, Ru, Rh, Pd, Ag, Cd, In, Lu, Ba, Hf, Ta, W, Os, Pt, Hg, La, Eu, Gd, Tb, Dy and Ho, in any oxidation state and in any combination thereof;

M′ represents at least one of VO₂, Ca, V, Cr, Mn, Fe, Co, Ni, Cu, Zn, Ga, Sr, Zr, Nb, Li, Na, K, Rb, Cs, Fr, Tl, Mo, Ru, Rh, Pd, Ag, Cd, In, Lu, Ba, Hf, Ta, W, Os, Pt, Hg, La, Eu, Gd, Tb, Dy and Ho, in any oxidation state and in any combination thereof; x is from 0.1 to about 1; y is from 0.1 to about 1; z is from 0.1 to about 4; and n is from 0.1 to about 24.

In preferred embodiments, A represents at least one of VO₂, Ca, V, Cr, Mn, Fe, Co, Ni, Cu, Zn, Ga, Sr, Zr, Nb, Li, Na, K, Rh, Cs, Fr, Tl, Mo, Ru, Rh, Pd, Ag, Cd, In, Lu, Ba, Hf, Ta, W, Os, Pt, Hg, La, Eu, Gd, Tb, Dy and Ho, in any oxidation state and in any combination thereof. In the further preferred embodiments, A represents at least one of Li, Na, K, Rb, Cs, and Fr, in any oxidation state and in any combination thereof. In other preferred embodiments, A represents Li, Na, K, Rb, in any oxidation state and in any combination thereof. In other preferred embodiments, A represents a mixture of K and other elements represented by A, where the molar ratio of K in the mixtures is at least 0.9, preferably, at least 0.95, most preferably at least 0.99. In the most preferred embodiments, A only represents K.

In some embodiments, B represents at least one of Cr, Mn, Fe, Eu, Gd, and Tb, in any oxidation state and in any combination thereof. In other preferred embodiments, B represents a mixture of Mn, Gd, and other elements represented by A, where the molar ratio of the combination of Mn and Gd in the mixtures is at least 0.9, preferably, at least 0.95, most preferably at least 0.99. In the most preferred embodiments, A represents a mixture of only Mn and Gd, in any oxidation state and in any combination thereof.

In some embodiments, M represents at least one of Fe, Co, and Ni, in any oxidation state and in any combination thereof. In some preferred embodiments, M represents only Fe. In still other embodiments, M′ represents at least one of Fe, Co, and Ni, in any oxidation state and in any combination thereof. In yet other embodiments, M′ represents only Fe. In preferred embodiments, each of M and M′, simultaneously, represents only Fe, in any oxidation state thereof.

As used herein, the term “in any combination thereof” for A, B, M, and M′ means that at least two of the elements that are represented by A, B, M, and M′ can be present in any molar ratios so long as the sum total is equal to the value for x, y, and z, and, in the case of M, the elements can be present in any molar ratios so long as the total amount of the M elements is equal to 4. Preferably, x in general formula (I) is from 0.2 to 0.9, more preferably from 0.3 to 0.8, more preferably still from 0.4 to 0.7, and most preferably from 0.5 to 0.6. Preferably, y in general formula (I) is from 0.2 to 0.9, more preferably from 0.3 to 0.8, more preferably still from 0.4 to 0.7, and most preferably from 0.5 to 0.6. Preferably, z in general formula (I) is from 0.2 to 3.5, more preferably from 0.3 to 0.8, more preferably still from 0.4 to 0.7, and most preferably from 0.5 to 0.6. All real numbers within the ranges for x, y, z, and n are included.

In some embodiments, a particularly preferred species of the Prussian blue compound represented by general formula (I) are K_{0.53}Gd_{0.89}Fe^{III}₄[Fe^{II}(CN)₆]_{3.8}·1.2H₂O and K_{0.6}Mn_{0.7}Fe^{III}₄[Fe^{II}(CN)₆]_{3.5}·3H₂O

In some embodiments, compositions disclosed herein may comprise Prussian blue materials that belong to the class of iron hexacyanoferrate (II). In some aspects, compositions disclosed herein may comprise Prussian blue materials represented by general formula (II):

Fe₄^{III}[Fe^{II}(CN)₆]₃·nH₂O (II)

wherein the value n represents an integer from 1 to about 24.

In other aspects, compositions disclosed herein may comprise Prussian blue salts represented by general formula (III):

A₄ₓFe₄₋ₓ^{III}[Fe^{II}(CN)₆]₃₊ₓ·nH₂O (III)

where A is an alkali metal such as lithium (Li+), sodium (Na⁺), Potassium (K⁺), Rubidium (Rb⁺), Cesium (Cs⁺), or it can be Ammonium (NH⁴⁺) or Thallium (Tl⁺). The value x can be any number, e.g. a fraction, from 0≤x≤1, e.g. 0.1 and n is about 1 to about 24, and preferably is from about 14 to about 16.

In some embodiments, compositions disclosed herein may comprise soluble Prussian blue materials insoluble Prussian blue materials. In an aspect, insoluble Prussian blue materials may be characterized by coordinating water molecules therein.

In various embodiments, compositions disclosed herein may comprise one or more metal isotopes doped to the Prussian blue materials described herein. In some aspects, a metal isotope may be Li, Na, K, Rb, Cs, Fr, Ga, In, Tl, Ca, Sc, V, Cr, Mn, Fe, Co, Ni, Cu, Zn, Sr, Y, Zr, Nb, Mo, Ru, Rh, Pd, Ag, Cd, Ba, La, Sm, Eu, Gd, Tb, Dy, Ho, Lu, Hf, Ta, W, Re, Os, Ir, Pt, Au, Hg, Pb, Bi or a combination thereof. In a preferred embodiment, the metal isotope may be Cs, Ga, Tl, In, V, Cr, Mn, Fe, Co, Ni, Cu, Mo, Ru, Ag, W, Pt, Au, Hg, Eu, Gd, or a combination therof.

In some embodiments, a metal isotope suitable for use in the compositions disclosed herein may be present in any sufficient oxidation state theoretically possible. In some aspects, a metal isotope may be Li(I), Na(I), K(I), Cs(I), Fr(I), Ga(III), In(III), Tl(I), Tl(III), Ca(II), Sc(III), V(III), V(IV), Cr(II), Cr(III), Mn(II), Mn(IV), Fe(II), Fe(III), Co(II), Co(III), Ni(II), Ni(III), Cu(I), Cu(II), Zn(II), Sr(II), Y(III), Zr(IV), Nb(IV), Nb(V), Mo(IV), Mo(V), Ru(III), Ru(IV), Rh(II), Rh(III), Rh(IV), Pd(II), Pd(IV), Ag(I), Cd(II), Ba(II), La(III), Sm(II), Sm(III), Eu(II), Eu(III), Gd(III), Tb(III), Tb(IV), Dy(III), Ho(III), Lu(III), Hf(IV), Ta(V), W(IV), W(V), Os(IV), Ir(II), Ir(III), Pt(II), Pt(IV), Au(I), Au(III), Hg(I), Hg(II), Pb(II), Pb(IV), Bi(III), or a combination thereof.

In various embodiments, a metal isotope suitable for use in the compositions disclosed herein may be linked to Prussian blue materials in a chemical or physical route. As a non-limiting example of chemical linkage, a metal isotope is bound by covalent bond, whereas the metal isotope replaces the Fe atom in the complex structure of a Prussian blue compound. In an aspect, Prussian blue materials may be represented by general formula (I):

AₓB_{y}M₄[M′(CN)₆]_{z}·nH₂O (I),

wherein M and M′ denote the same or different and independently from each other Cu-61, Cu-64, Cu-67, Zn-62, Zn-69m, Zn-69, Pb-206, Pb-207, Pb-208, Pb-209, Pb-210, Pb-211, Pb-212, Pb-213, Pb-214, Ag-105, Ag-106, Ag-112, Ag-113, Pt-186, Pt-187, Pt-188, Pt-190, Pt-191, Pt-197, La-131, La-132, La-133, La-135, La-140, La-141, La-142, Eu-150m, Eu-152m, Eu-158, Eu-145, Eu-146 and Eu-147, especially Cu-61, Cu-64, Cu-67, Ag-105, Ag-106, Ag-112, Ag-113, Pt-185, Pt-186, Pt-187, Pt-188, Pt-189, Pt-190, Pt-19 and Pt-197. A, B, y, x and n are as defined above. In a preferred embodiment, Prussian blue materials may be ng of Ag₄[Fe(CN)₆], Pb₂[Fe(CN)₆], Sn₂[Fe(CN)₆], Co[Cr(CN)₆]_{2/3} or a combination thereof.

As a non-limiting example of physical linkage, a metal isotope may be bounded by physical or physicochemical bonds, such as ion exchange, absorption, mechanical trapping. In an aspect, a metal isotope can be adsorbed on the surface of Prussian blue materials or incorporated into the vacancies of Prussian blue materials.

In various embodiments, compositions disclosed herein may comprise a metal isotope emitting any kind of radiation known in the field. In an aspect, the radiation may be alpha, beta, gamma, positron radiation, or a combination thereof. In some embodiments, compositions disclosed herein may comprise a metal isotope emitting alpha or beta radiation. In an aspect, a metal isotope emitting alpha or beta radiation may be Sc-47, Sc-48, Cu-67, Zn-69, Rb-86, Rb-84, Y-90, Zr-95, Zr-97, Nb-95, Nb-96, Nb-98, Ag-112, Ag-113, Cd-115, Cd-117, Cd-118, Cs-136, Cs-138, La-140, La-141, La-142, Sm-153, Eu-150m, Eu-152m, Eu-158, Tb-149, Dy-165, Dy-166, Ho-164, Ho-166, Ho-167, Hf-183, Ta-183, Ta-184, Ta-185, Re-186, Re-188, Re-189, Os-191, Os-193, Os-194, Os-195, Os-196, Ir-193, Ir-195, Pt-197, Pt-200, Au-196, Au-199, Hg-203, Hg-208, Pb-209, Pb-212, Bi-212, Bi-213, or a combination thereof.

In other embodiments, compositions disclosed herein may comprise a metal isotope emitting gamma or positron radiation. In an aspect, a metal isotope emitting gamma or positron radiation may be Sc-43, Sc-44, Cu-61, Cu-64, Zn-62, Zn-69m, Ga-67, Ga-68, Rb-81, Rb-82m, Y-84, Y-85, Y-86, Zr-86, Zr-87, Zr-88, Zr-89, Zr-90, Nb-88, Nb-89, Nb-90, Ag-105, Ag-106, Cd-104, Cd-105, Cd-107, Cd-111, Cs-127, Cs-129, Cs-131, Cs-134, Cs-135, La-131, La-132, La-133, La-135, Sm-141, Sm-142, Eu-145, Eu-146, Eu-147, Eu-152m, Tb-147, Tb-150, Tb-151, Tb-152, Tb-154, Tb-154m, Tb-156, Tb-156m, Dy-152, Dy-153, Dy-155, Dy-157, Ho-155, Ho-156, Ho-158, Ho-159, Ho-160, Ho-164, Hf-166, Hf-168, Hf-170, Hf-171, Hf-173, Hf-179, Ta-171, Ta-172, Ta-173, Ta-174, Ta-175, Ta-176, Ta-177, Ta-178, Re-181, Re-182, Re-183, Re-184, Re-186, Re-188, Re-190, Os-180, Os-181, Os-182, Os-183, Ir-183, Ir-184, Ir-185, Ir-186, Ir-187, Ir-188, Ir-189, Ir-190, Pt-185, Pt-186, Pt-187, Pt-188, Pt-189, Pt-190, Pt-191, Pt-197, Au-190, Au-191, Au-192, Au-193, Au-194, Au-196, Au-198, Au-199, Au-200, Au-201, Hg-190, Hg-191, Hg-193, Hg-197, Tl-194, Tl-195, Tl-196, Tl-197, Tl-198, Tl-199, Tl-200, Tl-201, Tl-202, Tl-203, Tl-204, Pb-206, Pb-207, Pb-208, Pb-209, Pb-210, Pb-211, Pb-212, Pb-213, Pb-214, Bi-200, Bi-201, Bi-201, Bi-203, Bi-204, Bi-205, Bi-206, or a combination thereof.

In various embodiments, compositions disclosed herein may comprise soluble Prussian blue materials forming a particle. In some aspects, a particle formed of Prussian blue materials may be a nanoparticle. In other aspects, a particle formed of Prussian blue materials may be a microparticle. In still other aspects, a particle formed of Prussian blue materials may be about 1 nanometer (nm) to about 10 microns (μm). In some embodiments, a particle formed of Prussian blue materials for use herein can range from about 10 nm to about 300 nm (e.g., about 10 nm, about 25 nm, about 50 nM, about 75 nM, about 100 nm, about 125 nm, about 150 nm, about 175 nm, about 200 nm, about 225 nm, about 250 nM, about 275 nM, about 300 nm) in diameter.

In various embodiments, compositions disclosed herein may comprise soluble Prussian blue materials synthesized by methods known in the art. In some aspects, a starting material can be a commercially available Prussian blue particle. In some aspects, a starting material may be commercially available from Radiogardase (by Heyltex). In another aspect, starting material can be a Prussian blue particle synthesized from FeCl₃ and K₄[Fe(CN)₆] which may be acidified for example with organic or inorganic acids (such as HCl, citric acid etc.) which is mixed. In some aspects, the step of mixing the solution may be temperature and pH controlled. In some aspects, a temperature suitable for mixing the solution disclosed herein may be about 20° C., about 21° C., about 22° C., about 23° C., about 24° C., about 25° C., or about 26° C. In some aspects, a pH suitable for mixing the solution disclosed herein may be about 3, about 4, about 5, about 6, or about 7. In other aspects, one or more additives known in the field to aid in foimation of Prussian blue particles with homogeneous size distribution and/or subsequent incorporation of a metal isotope and/or covering Prussian blue particles with a biocompatible coating as disclosed herein may be added during mixing of the solution disclosed herein.

In some embodiments, Prussian blue materials are synthesized by reacting a metallic salt with a metal cyanide ([M′(CN)₆]³⁻) in a solvent. In some aspects, the metallic salt comprises, consists essentially of, or consists of a salt of salt of V, Cr, Mn, Fe, Co, Ni, Cu, Zn, Ga, Sr, Zr, Nb, Li, Na, K, Rb, Cs, Fr, Tl, Mo, Ru, Rh, Pd, Ag, Cd, In, Lu, Ba, Hf, Ta, W, Os, Pt, Hg, La, Eu, Gd, Tb, Dy and Ho in any oxidation state thereof and in any combination thereof. In other aspects, the metallic salt comprises, consists essentially of, or consists of a metallic salt of a chloride, a nitrate, a nitrite, a sulfate, a fluorinate, a glutamate, an acetate, a carbonate, a citrate, a phosphate, a sulfate and any combination thereof. In still other aspects, the metal cyanide comprises, consists essentially of, or consists of a metal cyanide represented by [M′(CN)₆]³⁻, wherein M′ represents V, Cr, Mn, Fe, Co, Ni, Cu, Zn, Ga, Sr, Zr, Nb, Li, Na, K, Rb, Cs, Fr, Tl, Mo, Ru, Rh, Pd, Ag, Cd, In, Lu, Ba, Hf, Ta, W, Os, Pt, Hg, La, Eu, Gd, Tb, Dy and Ho in any oxidation state thereof and in any combination thereof.

In some embodiments, the solvent in which the reaction between the metallic salt and the metallic cyanide described above occurs may not be particularly limited, so long as the reaction proceeds in this solvent. In some embodiments, the solvent comprises, consists essentially of, or consists of water, air, or an organic solvent. In some embodiments, the solvent is ultrapure water. As used herein, the “ultrapure water” refers to “grade 1” water as defined by the International Organization for Standardization (ISO), with resistivity of 18.2 MΩ·cm. As used herein, the terms “ultrapure water” and “Milli-Q water” are synonymous.

In some embodiments, the solvent in which the reaction between the metallic salt and the metallic cyanide described above occurs is an organic solvent. In some embodiments, the organinc solvent can be hydrophilic to any degree or hydrophobic to any degree. In a preferred aspect, the organic solvent comprises, consists essentially of, or consists of hexane; benzene; toluene; diethyl ether; chloroform; 1,4-dioxane; ethyl acetate; tetrahydrofuran (THF); dichloromethane; acetone; acetonitrile (MeCN); dimethylformamide (DMF); dimethyl sulfoxide (DMSO); a polar protic solvent; acetic acid; n-butanol; isopropanol; n-propanol; ethanol; methanol; formic acid; and any combination thereof, so long as the metallic salt and the metallic cyanide are sufficiently dissolved in the combination and the reaction proceeds in this combination of solvents.

### (i) Biocompatible Coatings

In certain embodiments, PBNP compositions disclosed herein may comprise a shell partially or completely encapsulating a nanoparticle. In some aspects, a shell encapsulates about 25%, about 50%, about 75%, or about 100% of the nanoparticle. In some embodiments, a shell ay completely encapsulates a nanoparticle formed of Prussian blue materials as disclosed herein.

In some embodiments, a shell is comprised of a biocompatible coating. In some embodiments, a biocompatible coating comprises one or more biocompatible materials assisting to in vivo and in vitro use of compositions disclose herein. In some embodiments, a biocompatible coating of the shell may comprise at least one material selected from the group consisting of dextran; chitosan; silica; polyethylene glycol (PEG); avidin; a proteins; a nucleic acids; a carbohydrates; a lipid; neutravidin; streptavidin; gelatin; collagen; fibronectin; albumin; a serum protein; lysozyme; a phospholipid; a polyvinyl pyrrolidone (PVP); a polyvinyl alcohol; a polyethylene glycol diacrylate; polyethylenimine (PEI); and a combination thereof. Without wishing to be bound to any particular theory, the biocompatible coating is believed to prevent the compositions from aggregating and to prevent leakage of ions from the core to the surrounding environment.

In some embodiments, a dextran of the biocompatible coating may comprise a dextran that is a complex, branched polysaccharide having chains of varying lengths, preferably chains having lengths of from about 3 to about 2000 kDa. In other embodiments, a chitosan of the biocompatible coating may comprise a linear polysaccharide having randomly distributed units of β-(1-4)-linked D-glucosamine (deacetylated unit) and units of N-acetyl-D-glucosamine (acetylated unit). In still other embodiments, a silica of the biocompatible coating may comprise an oxide of silicon with the chemical formula SiO₂. In yet other embodiments, a polyethylene glycol (PEG) of the biocompatible coating may comprise polyethylene oxide (PEO) or polyoxyethylene oxide (POE). In other embodiments, an avidin of the biocompatible coating may comprise a protein produced in the oviducts of birds, reptiles and amphibians deposited in the whites of their eggs. In yet other embodiments, an albumin of the biocompatible coating may comprise bovine serum albumin (BSA, fraction V), human serum albumin (HSA) and all serum albumin derived from mammals. In an aspect, serum proteins of the biocompatible coating may comprise at least one member selected from the group consisting of Orosomucoid; antitrypsin; alpha-1 antichymotrypsin; alpha-2 macroglobulin (AMG); haptoglobin; transferrin; beta lipoprotein (LDL); immunoglobulin A (IgA); immunoglobulin M (IgM); immunoglobulin G (IgG); immunoglobulin E (IgE); and immunoglobulin D (IgD). in some embodiments, a lysozyme of the biocompatible coating may be of N-acetylmuramide glycanhydrolase. In still other embodiments, phospholipids of the biocompatible coating may comprise of all natural phospholipids and synthetic phospholipids. Non-limiting examples of natural phospholipids and synthetic phospholipids include DMPA, DPPA, DSPA DDPC, DLPC, DMPC, DPPC, DSPC, DOPC, POPC, DEPC DMPG, DPPG, DSPG, POPG DMPE, DPPE, DSPE DOPE DOPS mPEG-phospholipid, polyglycerin-phospholipid, functionalized-phospholipid, and terminal activated-phospholipid. In other embodiments, a polyvinyl pyrrolidone (PVP) of the biocompatible coating may comprise a polymer made from repeating monomer N-vinylpyrrolidone units. In an aspect, the molecular weight of the PVP is not particularly limited, as long as the PVP is suitable for use in the biocompatible coating of the present disclosure. As used herein, the terms “polyvidone” and “povidone” are synonymous with PVP. In yet other embodiments, a polyvinyl alcohol of the biocompatible coating may comprise PVOH, PVA, and PVAI. In an aspect, molecular weights of the PVOH, PVA, and PVAI are not particularly limited, as long as the PVOH, PVA, and PVAI are suitable for use in the biocompatible coating of the present disclosure. In other embodiments, a polyethylene glycol diacrylate of the biocompatible coating may comprise a polyethylene glycol terminated with acrylate groups. In an aspect, molecular weight of the polyethylene glycol diacrylate is not particularly limited, as long as the polyethylene glycol diacrylate is suitable for use in the biocompatible coating of the present disclosure. In some other embodiments, lipids of the biocompatible coating may comprise sterols, fats, oils, waxes, vitamin A, vitamin D, vitamin E, vitamin K, phospholipids of claim 5q, (mono-, di-, tri-) glycerides, or a combination thereof.

In preferred embodiments, a biocompatible coating of the shell of PBNP compositions disclosed herein may comprise one or more polymers. In an aspect, a polymer suitable for use in a biocompatible coating disclosed herein may be polyethylene glycol, polypropylene glycol, polyoxyethylene ether, polyanethol sulfonic acid, polyethylene imine, polymaleimide, polyvinyl alcohol, polyvinyl chloride, polyvinyl acetate, polyvinyl pyrrolidone, polyvinyl sulfate, polyacrylic acid, polymethacrylic acid, polylactide, polylactide glycide, or a combination thereof. In a perfered aspect, the biocompatible coating comprises polyethylene imine (PEI).

In some embodiments, the biocompatible coating can be applied to the core of the compositions disclosed herein by a variety of physical and chemical interactions including, but not limited to: electrostatic (charge-based), covalent, hydrophobic and van der Waal's interactions. In some embodiments, the biocompatible coating is applied by suspending the core in a solution comprised of one or more materials selected from the group consisting of dextran; chitosan; silica; polyethylene glycol (PEG); avidin; a proteins; a nucleic acids; a carbohydrates; a lipid; neutravidin; streptavidin; gelatin; collagen; fibronectin; albumin; a serum protein; lysozyme; a phospholipid; a polyvinyl pyrrolidone (PVP); a polyvinyl alcohol; a polyethylene glycol diacrylate; polyethylenimine (PEI); and a combination thereof.

### (ii) Biomolecules

In certain embodiments, PBNP compositions disclosed herein may comprise a shell partially or completely encapsulating a nanoparticle with biocompatible coating wherein at least one biomolecule may be attached to, or absorbed to, the biocompatible coating. In a preferred aspect, the shell completely encapsulates a nanoparticle formed of Prussian blue materials with biocompatible coating wherein at least one biomolecule may be attached to, or absorbed to, the biocompatible coating.

In come embodiments, a biocompatible coating disclosed herein may absorb at least 25%, at least 50%, or at least 75% biomolecule weight by total weight of the biocompatible coating. In other embodiments, at least 25%, at least 50%, at least 75%, at least 100% of the outer surface of the biocompatible coating has biomolecules attached.

In various embodiments, a biomolecule attached to, or absorbed to, the biocompatible coating may comprise an antibody, a peptide, a protein, an enzyme, an amino acid, a nucleic acid, a carbohydrate, a fat, an aptamer, a small molecule, a synthetic molecule or a combination thereof.

In some embodiments, a nucleic acid may be DNA (deoxyribonucleic acid), RNA (ribonucleic acid), a peptide nucleic acid, a morpholino-nucleic acid, a locked nucleic acid, a glycol nucleic acid, a threose nucleic acid, an oligonucleotide, or a combination thereof. In some embodiments, the biomolecule may be an oligonucleotide. In an aspect, an oligonucleotide may be at least 5, at least 10, at least 25, at least 50, at least 75, at least 100, at least 250, or at least 500 base pairs (bp). In another aspect, the oligonucleotide may be an oligodeoxynucleotide.

In some embodiments, at least one of the biomolecules may be an antibody. As used. herein, an “antibody” or “antibody molecule” is any immunoglobulin, including antibodies and fragments thereof, that binds to a specific antigen. The telin includes polyclonal, monoclonal, chimeric, single domain (Dab) and bispecific antibodies. As used herein, antibody or antibody molecule contemplates recombinantly generated intact immunoglobulin molecules and immunologically active portions of an immunoglobulin molecule such as, without limitation: Fab, Fab′, F(ab′)₂, F(v), scFv, scFv₂, scFv-Fc, minibody, diabody, tetrabody, single variable domain (e.g., variable heavy domain, variable light domain), bispecific, and peptabodies.

In other embodiments, at least one of the biomolecules may be a peptide. In some embodiments, a peptide may consist of any sequence of 50 amino acids or less, excluding zero. In another aspect, a peptide may consist of any sequence of about 2 amino acids to about 50 amino acids. In a preferred aspect, a peptide may consist of any sequence of 20 amino acids or less, excluding zero. As used herein, “amino acids” are represented by their full name, their three letter code, or their one letter code as well known in the art. Amino acid residues are abbreviated as follows: Phenylalanine is Phe or F; Leucine is Leu or L; Isoleucine is Ile or I; Methionine is Met or M; Valine is Val or V; Serine is Ser or S; Proline is Pro or P; Threonine is Thr or T; Alanine is Ala or A; Tyrosine is Tyr or Y; Histidine is His or H; Glutamine is Gln or Q; Asparagine is Asn or N; Lysine is Lys or K; Aspartic Acid is Asp or D; Glutamic Acid is Glu or E; Cysteine is Cys or C; Tryptophan is Trp or W; Arginine is Arg or R; and Glycine is Gly or G. An amino acid as disclosed herein may be either naturally or non-naturally occurring. As used herein, a “naturally occurring amino acid” is one that has the general core structure

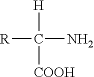

and that is synthesized in nature. of naturally occurring amino acids that may be used in the present disclosure include, but are not limited to, alanine, arginine, asparagine, aspartic acid, carnitine, cysteine, glutamine, glutamic acid, glycine, citrullline, histidine, isoleucine, leucine, lysine, methionine, phenylalanine, proline, serine, threonine, tryptophan, tyrosine, and derivatives, analogs, and combinations thereof. The present disclosure may include levorotary (L) stereoisomers of such amino acids.

As used herein, a “non-naturally occurring amino acid” may be an analog, derivative and/or enantiomer of a naturally occurring amino acid. The term “non-naturally occurring amino acid” includes, but is not limited to, amino acids that occur naturally by modification of a naturally encoded amino acid (including but not limited to, the 20 naturally occurring amino acids contained in body proteins or pyrrolysine and selenocysteine) but are not themselves incorporated into a growing polypeptide chain by the translation complex. Non-limiting examples of non-naturally occurring amino acids that may be used in the present disclosure include L-hydroxypropyl, L-3,4-dihydroxyphenylalanyl, alpha-amino acids such as L-alpha-hydroxylysyl and D-alpha-methylalanyl, L-alpha-methylalanyl, beta-amino acids, N-acetylglucosaminyl-L-serine, N-acetylglucosaminyl-L-threonine, O-phosphotyrosine, and isoquinolyl.

As used herein, the term “amino acid” may also encompass chemically modified amino acids, including but not limited to salts, amino acid derivatives (such as amides), and substitutions. Amino acids contained within the compositions of the present disclosure can be modified by methylation, amidation, acetylation or substitution with other chemical groups.

In some embodiments, at least one of the biomolecules may be a protein. As used herein, a “protein” can be a macromolecule comprising 20 or more contiguous amino acid residues. In some embodiments, a protein may consist of any sequence of about 20 amino acids to about 40,000 amino acids, about 100 amino acids to about 20,000 amino acids, or about 200 amino acids to about 10,000 amino acids. In some aspects, a protein may consist of any sequence of about 20 amino acids, about 50 amino acids, about 100 amino acids, about 200 amino acids, about 500 amino acids, about 1,000 amino acids, about 5,000 amino acids, about 10,000 amino acids, about 20,000 amino acids, or about 40,000 amino acids. in preferred aspects, a protein may consist of any sequence of about 200 amino acids to about 400 amino acids. In some aspects, the protein may be a native protein. In other aspects, the protein may be a synthetic protein. In still other aspects, the protein may be a recombinant protein. In an aspect, a recombinant protein can be a chimeric protein, a fusion protein, a truncated protein or a combination thereof. In other aspects, the protein may be in the form of a miltimer (e.g., a dimer, trimer, tetramer, or oligomer).

In some embodiments, a biomolecule may further comprise one or more prodrugs. In some aspects, a prodrug suitable for use in compositions disclosed herein may be intracellular Type IA, such as Acyclovir, 5-fluorouracil, cyclophosphamide, diethylstilbestrol diphosphate, L-dopa, 6-mercaptopurine, mitomycin C, zidovudine; intracellular Type IB, such as carbamazepine, captopril, carisoprodol, heroin, molsidomine, paliperidone, phenacetin, primidone, psilocybin, sulindac, and fursultiamine; extracellular Type IIA, such as lisdexamfetamine, loperamide oxide, oxyphenisatin, and sulfasalazine; extracellular Type IIB, such as acetylsalicylate, bacampicillin, bambuterol, chloramphenicol succinate, dihydropyridine pralidoxime, dipivefrin, and fosphenytoin; and extracellular Type IIC, such as ADEPTS, GDEPs, VDEPs, or a combination thereof.

In some embodiments, PBNP compositions disclosed herein may comprise one or more immune signals biofunctionalized on the surface of the PBNPs. In some embodiments, one or more immune signals biofunctionalized on the surface of the PBNPs can include toll-like receptor (TLR) agonists, CD137 agonists, Rig agonists, PD-1 antagonists, CTLA-4 antagonists, TIM-3 antagonists, CD28 agonists, and the like.

### (iii) Photothermal Therapy

In some embodiments, the PBNP compositions disclosed herein may be used in photothermal therapy. As used herein, “photothermal therapy” is a method of accumulating a material generating heat by absorbing light in a location requiring hyperthermal therapy and irradiating light. Photothermal heating via incident light and PBNPs is termed PBNP-PTT in the present disclosure. Herein, the general principles underlying photothermal treatment (PTT) generally known by those skilled in the art are employed.

In some embodiments photothermal therapy may use a light source to irradiate PBNP compositions disclosed herein. In some embodiments, a PBNP composition disclosed herein may absorb near infrared radiation (NIR) delivered thereto, thus becoming irradiated. Devices and methods for delivering radiation of a particular wavelength include, but not limited to, lasers well-known and standard in the art. In an aspect, the amount of light delivered to a cell via PTT may be determined based on the physical dimensions and thermal characteristics of the tissue to be treated, such that the absorption of said light leads to the desired temperature increase in the tissue. In some embodiments, a method of using PBNP compositions disclosed herein for use in photothermal therapy further comprises calculating output power of the laser based at least in part upon one of heat dissipation and conductivity values within the cell culture or shape factor values of the t cell culture and/or determining time of exposure of the laser. In some embodiments, the light wavelength is in a range of about 600 to about 1500 nm (e.g., about 600 nM, about 650 nM, about 700 nm, about 750 nM, about 800 nm, about 850 nM, about 900 nm, about 950 nM, about 1000 nm, about 1500 nm). In preferred embodiments, the light wavelength can range from about 650 nm to about 900 nm.

In some embodiments, PBNP compositions disclosed herein may be irradiated after exposure to a light source for about 4 minutes to 20 minutes. In other embodiments, PBNP compositions disclosed herein may be irradiated after exposure to a light source for about 4 minutes, about 6 minutes, about 8 minutes, about 10 minutes, about 12 minutes, about 14 minutes, about 16 minutes, about 18 minutes, or about 18 minutes. In some embodiments, PBNP compositions disclosed herein may be irradiated after exposure to NIR for about 10 minutes.

In some embodiments, a method of irradiating PBNP compositions disclosed herein by administering photothermal therapy can result in a thermal interaction at the site of the targeted cell. In some aspects, a thermal interaction may increase temperatures of the targeted cell to at least about 40° C., at least about 41° C., at least about 42° C., at least about 43° C., or at least about 44° C. In other aspects, a thermal interaction may increase temperatures of the targeted cell to stimulate cell and/or tissue death. In yet other aspects, a thermal interaction may increase temperatures of the targeted cell to stimulate cell immune response. In some embodiments, a thermal interaction may increase temperatures of the targeted cell to stimulate cell immune response by at least a 2-fold, at least a 5-fold, at least a 10-fold, at least a 20-fold, at least a 50-fold compared to an untreated cell.

In some embodiments, a method of irradiating PBNP compositions disclosed herein by administering PTT can result a cellular and/or tissue cytotoxic T lymphocyte response. As used herein, a “cytotoxic T lymphocyte response” or “CTL response” refers to an immune response in which cytotoxic T cells are activated by photothermal therapy. A CTL response can include the activation of precursor CTLs as well as differentiated CTLs. In some aspects, a CTL response may include any measurable CTL response for at least one CTL that is specific for an antigen expressed on an autologous tumor cell. In some embodiments, a method of irradiating PBNP compositions disclosed herein by administering PTT may result in an increased the frequency of precursor CTLs specific for tumor antigens compared to an untreated cell. In some embodiments, a method of irradiating PBNP compositions disclosed herein by administering PTT may increase the frequency of precursor CTLs specific for tumor antigens by at least about 2-fold, at least about 5-fold, at least about 10-fold, at least about 20-fold, at least about 50-fold compared to an untreated cell. In some embodiments, a method of irradiating PBNP compositions disclosed herein by administering PTT may stimulate the frequency of CTLs for tumor cells compared to an untreated cell. In some embodiments, a method of irradiating PBNP compositions disclosed herein by administering PTT may stimulate the frequency of CTLs for tumor cells by at least about 2-fold, at least about 5-fold, at least about 10-fold, at least about 20-fold, at least about 50-fold compared to an untreated cell. In some embodiments, a method of irradiating PBNP compositions disclosed herein by administering PTT may result in T cell proliferation compared to an untreated cell. In some embodiments, a method of irradiating PBNP compositions disclosed herein by administering PTT may increase T cell proliferation by at least about 50%, at least about 60%, at least about 70%, at least about 80%, or at least about 90%.

In some embodiments, a method of irradiating PBNP compositions disclosed herein by administering PTT may be applied to target cells herein to generate target cell immunogenicity. As used herein, the term “immunogenicity” refers to the ability of cells/tissues to provoke an immune response. In some embodiments, a method of irradiating PBNP compositions disclosed herein by administering PTT may be applied to target cells herein to generate target cell immunogenicity as measured by one or more biochemical correlates of immunogenic cell death (ICD). Examples of biochemical correlates of ICD include, but are not limited to, ATP, calreticulin, HMGB1, and the like. Other cell markers of immunogenicity suitable for use herein can include, but not limited to, co-stimulatory molecule expression, co-inhibitory molecule expression, immune checkpoint expression, MHC expression, and/or antigen release.

### (c) Cytokines

In some embodiments, target cancer type specificity in an immune cell disclosed herein can be modulated by exposure to one or more cytokines for the duration of ex vivo expansion. In some aspects, cytokines suitable for ex vivo expansion as disclosed herein may be interferon gamma (IFN-γ), tumor necrosis factor-alpha (TNF-α), interleukin-2 (IL-2), interleukin-12 (IL-12), type I interferons, interferon alpha (INF-α), interferon beta (INF-β), granulocyte-macrophage colony-stimulating factor (GM-CSF), interleukin 1 alpha (IL-1α), interleukin-4 (IL-4), interleukin-6 (IL-6), interleukin-7 (IL-7), interleukin-9 (IL-9), interleukin-15 (IL-15), interleukin-18 (IL-18), interleukin-21 (IL-21), leukemia inhibitory factor (LIF), or a combination thereof. In some embodiments, cytokines suitable for ex vivo expansion as disclosed herein may IL-2, IL-15, IL-7, IL-12, IL-18, IL-21 or a combination thereof.

In some embodiments, a cytokine in an amount of about 1 ng/ml to about 1000 μg/ml, about 10 ng/ml to about 750 μg/ml, or about 25 ng/ml to about 500 μg/ml may be added for the duration of ex vivo expansion as disclosed herein. In some embodiments, a cytokine in an amount of about 1 ng/ml, about 10 ng/ml, about 25 ng/ml, about 50 ng/ml, about 75 ng/ml, about 100 ng/ml, about 250 ng/ml, about 500 ng/ml, about 750 ng/ml, about 1 μg/ml, about 10 μg/ml, about 25 μg/ml, about 50 μg/ml, about 75 μg/ml, about 100 μg/ml, about 250 μg/ml, about 500 μg/ml, about 750 μg/ml, or about 1000 μg/ml may be added during ex vivo expansion as disclosed herein.

### (d) Immune Cell Therapy

In certain embodiments, an immune cell therapy composition disclosed herein can include at least one immune cell with modulated gene expression. As used herein, the term “immune cell therapy” or “immunotherapy” refers to a therapeutic approach of activating or suppressing the immune system for the treatment of disease. In some embodiments, an immune cell therapy composition disclosed herein encompasses adoptive cell therapy. As used herein, the term “adoptive cell therapy” refers to the transfer of ex vivo grown immune cells into a subject for treatment of a disease.

In certain embodiments, immune cell therapy compositions disclosed herein include ex vivo expanded immune cells having specificity to at least one target cell. In some embodiments, immunotherapy compositions disclosed herein may be ex vivo expanded immune cells having specificity to at least one target cancer cell. In some aspects, immunotherapy compositions disclosed herein can be a cytotoxic lymphocyte having specificity to at least one target cancer cell. In some other aspects, immunotherapy compositions disclosed herein can be a a CD4 T cell and/or a CD8 T cell having specificity to at least one target cancer cell.

In certain embodiments, immunotherapy compositions disclosed herein can be administered to a subject in need thereof. A suitable subject includes a mammal, a human, a livestock animal, a companion animal, a lab animal, or a zoological animal. In some embodiments, the subject may be a rodent, e.g., a mouse, a rat, a guinea pig, etc. In another embodiment, the subject may be a livestock animal. Non-limiting examples of suitable livestock animals may include pigs, cows, horses, goats, sheep, llamas and alpacas. In yet other embodiments, the subject may be a companion animal. Non-limiting examples of companion animals may include pets such as dogs, cats, rabbits, and birds. In yet another embodiment, the subject may be a zoological animal. As used herein, a “zoological animal” refers to an animal that may be found in a zoo. Such animals may include non-human primates, large cats, wolves, and bears. In a specific embodiment, the animal is a laboratory animal. Non-limiting examples of a laboratory animal may include rodents, canines, felines, and non-human primates. In certain embodiments, the animal is a rodent. Non-limiting examples of rodents may include mice, rats, guinea pigs, etc. In preferred embodiments, the subject is a human.

In some embodiments, a subject in need thereof may have been diagnosed with a cancer. By example, but not limited to, a subject may have been diagnosed with nasopharyngeal cancer, synovial cancer, hepatocellular cancer, renal cancer, cancer of connective tissues, melanoma, lung cancer, bowel cancer, colon cancer, rectal cancer, colorectal cancer, brain cancer, throat cancer, oral cancer, liver cancer, bone cancer, pancreatic cancer, choriocarcinoma, gastrinoma, pheochromocytoma, prolactinoma, T-cell leukemia/lymphoma, neuroma, von Hippel-Lindau disease, Zollinger-Ellison syndrome, adrenal cancer, anal cancer, bile duct cancer, bladder cancer, ureter cancer, brain cancer, oligodendroglioma, neuroblastoma, meningioma, spinal cord tumor, bone cancer, osteochondroma, chondrosarcoma, Ewing's sarcoma, cancer of unknown primary site, carcinoid, carcinoid of gastrointestinal tract, fibrosarcoma, breast cancer, Paget's disease, cervical cancer, colorectal cancer, rectal cancer, esophagus cancer, gall bladder cancer, head cancer, eye cancer, neck cancer, kidney cancer, Wilms' tumor, liver cancer, Kaposi's sarcoma, prostate cancer, lung cancer, testicular cancer, Hodgkin's disease, non-Hodgkin's lymphoma, oral cancer, skin cancer, mesothelioma, multiple myeloma, ovarian cancer, endocrine pancreatic cancer, glucagonoma, pancreatic cancer, parathyroid cancer, penis cancer, pituitary cancer, soft tissue sarcoma, retinoblastoma, small intestine cancer, stomach cancer, thymus cancer, thyroid cancer, trophoblastic cancer, hydatidiform mole, uterine cancer, endometrial cancer, vagina cancer, vulva cancer, acoustic neuroma, mycosis fungoides, insulinoma, carcinoid syndrome, somatostatinoma, gum cancer, heart cancer, lip cancer, meninges cancer, mouth cancer, nerve cancer, palate cancer, parotid gland cancer, peritoneum cancer, pharynx cancer, pleural cancer, salivary gland cancer, tongue cancer, tonsil cancer, or a combination thereof.

In some embodiments, immunotherapy compositions disclosed herein may have increased cytolytic activity compared to cytolytic activity of native immune cells. In some aspects, an immune cell therapy composition disclosed herein may have an increase in cytolytic activity by about 1% to about 100%, about 10% to about 90%, or about 20% to about 80% compared to native immune cells. In other aspects, an immune cell therapy composition disclosed herein may an increase in cytolytic activity by about 1%, about 5%, about 10%, about 20%, about 30%, about 40%, about 50%, about 60%, about 70%, about 80%, about 90%, or about 100% compared to native immune cells. In still other aspects, an immune cell therapy composition disclosed herein may increase cytolytic activity against leukemia cells, lymphoma cells, tumor cells, metastasizing cells of solid tumors compared to cytolytic activity of native immune cells.

### (e) Pharmaceutical Compositions

In certain embodiments, pharmaceutical compositions are provided herein. The pharmaceutical compositions contemplated I the present disclosure can contain a pharmaceutically acceptable carrier combined with a target cell specific immune cell as described herein. Pharmaceutically acceptable excipients (carriers) are well known in the art.

In certain embodiments, an immune cell therapy composition disclosed herein can be formulated for parenteral administration by injection. In some aspects, parenteral administration by injection can be by infusion. In some embodiments, immune cell therapy formulations disclosed herein can encompasses a combination of ex vivo expanded immune cells as disclosed herein and at least one additional component selected from the group consisting of pharmaceutically acceptable excipients, adjuvants, diluents, preservatives, antibiotics, and combinations thereof.

In some embodiments, immune cell therapy formulations disclosed herein may be formulated in a conventional manner using one or more physiologically acceptable carriers including excipients and auxiliaries which can facilitate processing of active components into preparations which can be used pharmaceutically. In other embodiments, proper formulation of immune cell therapy formulations disclosed herein may be dependent upon the route of administration chosen. In an aspect, any of the well-known techniques, carriers, and excipients may be used as suitable and as understood in the art. A summary of carriers, and excipients suitable for use in immune cell therapy formulations described herein may be found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pa. 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999), herein incorporated by reference in their entirety for such disclosure.

“Adjuvants” as used herein are agents that enhance the immune response of an antigen. In some embodiments, one or more adjuvants may be a particulate adjuvant In some embodiments, one or more adjuvants may be an emulsion. In some embodiments, one or more adjuvants may be a water-in-oil emulsion, oil-in-water emulsion, water-in-oil-in-water emulsion. The emulsion can be based in particular on light liquid paraffin oil (European Pharmacopea type); isoprenoid oil such as squalane or squalene oil resulting from the oligomerization of alkenes, in particular of isobutene or decene; esters of acids or of alcohols containing a linear alkyl group, more particularly plant oils, ethyl oleate, propylene glycol di-(caprylate/caprate), glyceryl tri-(caprylate/caprate) or propylene glycol dioleate; esters of branched fatty acids or alcohols, in particular isostearic acid esters. The oil can be used in combination with emulsifiers to form the emulsion. The emulsifiers may be nonionic surfactants, in particular esters of sorbitan, of mannide (e.g. anhydromannitol oleate), of glycol, of polyglycerol, of propylene glycol and of oleic, isostearic, ricinoleic or hydroxystearic acid, which are optionally ethoxylated, and polyoxypropylene-polyoxyethylene copolymer blocks, in particular the Pluronic products, especially L121. See Hunter et al., The Theory and Practical Application of Adjuvants (Ed.Stewart-Tull, D. E. S.). JohnWiley and Sons, NY, pp51-94 (1995) and Todd et al., Vaccine 15:564-570 (1997). In some embodiments, one or more adjuvants may be a liposome. In some embodiments, one or more adjuvants may be a microsphere of biodegradable polymers. In some embodiments, one or more adjuvants may be an immunomodulator. In some embodiments, an adjuvant system of the present disclosure may be any combination of adjuvants and immunomodulators. Non-limiting examples of immunomodulators comprise monophosphoryl lipid A, bark-saponin Quil A, dsRNA analogues, and N-acetyl muramyl-L-alanyl-D-isoglutamine. Further suitable adjuvant systems useful to the present disclosure include, but are not limited to, the RIBI adjuvant system (Ribi Inc.), Block co-polymer (CytRx, Atlanta GA), SAF-M (Chiron, Emeryville CA), AS15, MF59, Avridine lipid-amine adjuvant, heat-labile enterotoxin from E. coli (recombinant or otherwise), cholera toxin, IMS 1314, GLA-SE, IC31, CAF01, ISCOMs, or muramyl dipeptide among many others.

In some embodiments, immune cell therapy formulations disclosed herein that are formulations for injection may be presented in unit dosage form. In some aspects, a unit dosage form may be in ampoules and or in multi-dose containers. In other aspects, immune cell therapy formulations disclosed herein may be suspensions, solutions or emulsions in oily or aqueous vehicles. In still other aspects, pharmaceutical compositions disclosed herein may contain formulatory agents such as suspending, stabilizing and/or dispersing agents. In other aspects, immune cell therapy formulations disclosed herein may be cryofrozen prior to storage. As used herein, “cryofrozen” refers to and/or describes cryopreservation biological samples frozen in a manner that maintains vitality and subsequently thawed out again as needed while maintaining vitality. In some aspects, immune cell therapy formulations disclosed herein may be cryofrozen and stored for up to 1 week, up to 4 weeks, up to 8 weeks, up to 16 weeks, up to 25 weeks, up to 50 weeks, up to 100 weeks, or up to 200 weeks while maintaining vitality.

In some embodiments, immune cell therapy formulations described herein for parenteral administration can include aqueous and non-aqueous (oily) sterile injection solutions of the compositions which may contain antioxidants, buffers, bacteriostats and solutes which render the formulation isotonic with the blood of the intended recipient; and aqueous and non-aqueous sterile suspensions which may include suspending agents and thickening agents. In some aspects, immune cell therapy formulations described herein may include lipophilic solvents or vehicles. Non-limiting examples of vehicles include fatty oils such as sesame oil, or synthetic fatty acid esters, such as ethyl oleate or triglycerides, or liposomes. In some embodiments, immune cell therapy formulations described herein may be aqueous injection suspensions. In some aspects, immune cell therapy formulations described herein may contain substances which increase the viscosity of the suspension, such as sodium carboxymethyl cellulose, sorbitol, or dextran. In other aspects, immune cell therapy formulations described herein may comprise suitable stabilizers or agents which increase the solubility of the enzymes and fining agents to allow for the preparation of highly concentrated solutions.

### (II) Immune Cell Expansion Kits

In some embodiments, the present disclosure provides kits including at least one or more compositions disclosed herein for ex vivo expansion of immune cells. In other embodiments, the present disclosure provides packaging including at least one or more compositions disclosed herein for ex vivo expansion of immune cells.

The present disclosure may further encompass a kit, wherein the kit includes at least PBNP as described herein. In various embodiments, a kit may further include at least one culture medium. In some aspects, the culture medium can be an initial culture medium. In other aspects, the culture medium can be a subculture medium. In various embodiments, a kit can be used for ex vivo expansion of isolated immune cells. In some aspects, a kit can be used for generating target cell specific immune cells from isolated immune cells during ex vivo immune cell expansion. In other aspects, a kit can be used to increase rate of ex vivo expansion in isolated immune cells during ex vivo immune cell expansion. In still other aspects, a kit can be used to enhance immune cell activation in isolated immune cells during ex vivo immune cell expansion. In yet other aspects, a kit can be used to increase cytolytic activity in isolated immune cells during ex vivo immune cell expansion.

In some embodiments, a kit can be used for ex vivo expansion of isolated immune cells wherein the kit does not require use of feeder cells. In some embodiments, a kit can be used for ex vivo expansion of isolated immune cells wherein the kit does require use of feeder cells. As used herein, the term “feeder cells” refers to a layer of cells that provide extracellular secretions and/or structure to help an isolated immune cell to proliferate during ex vivo expansion. Non limiting examples of feeder cells can include HeLa cells, 3T3 cells, human dermal fibroblasts, adipose-derived mesenchymal stem cells, human bone marrow-derived mesenchymal cells, mouse embryonic fibroblasts, human fetal muscle cells, human fetal fibroblasts, human adult fallopian tubal epithelial cells, human amniotic mesenchymal cells, human amniotic epithelial cells, mouse bone marrow stromal cells, and murine amniocytes.

In some embodiments, a kit can be used to expand immune cells as disclosed herein by at least about 1-fold to about 500-fold, about 10-fold to about 400-fold, or about 50-fold to about 300-fold. In some aspects, immune cells as disclosed herein can be expanded by about 1-fold, about 10-fold, about 50-fold, about 100-fold, about 150-fold, about 200-fold, about 250-fold, about 300-fold, about 350-fold, about 400-fold, about 450-fold, or about 500-fold.

In some embodiments, a kit can be used to expand immune cells as disclosed herein within about 1 week to about 6 weeks of culture, about 2 weeks to about 5 weeks of culture, or about 3 weeks to about 4 weeks of culture. In some aspects, a kit can be used to immune cells as disclosed herein within about 1 week, about 2 weeks, about 3 weeks, about 4 weeks, about 5 weeks, or about 6 weeks of culture.

In some embodiments, a kit can further include at least one cytokine. In some aspects, cytokines can be interferon gamma (IFN-γ), tumor necrosis factor-alpha (TNF-α), interleukin-2 (IL-2), interleukin-12 (IL-12), type I interferons, interferon alpha (INF-α), interferon beta (INF-β), granulocyte-macrophage colony-stimulating factor (GM-CSF), interleukin 1 alpha (IL-1α), interleukin-4 (IL-4), interleukin-6 (IL-6), interleukin-7 (IL-7), interleukin-9 (IL-9), interleukin-15 (IL-15), interleukin-18 (IL-18), interleukin-21 (IL-21), leukemia inhibitory factor (LIF), or a combination thereof. In preferred aspects, cytokines suitable use in kits disclosed herein may IL-2, IL-15, IL-7, IL-12, IL-18, IL-21 or a combination thereof.

In various embodiments, a kit may further comprise instructions for methods of use. In some aspects, instructions to be provided with a kit may be in a fixed form. Non-limiting examples of fixed form instructions include written, recorded onto an audiocassette, videocassette, compact disc, or digital videodisc. In other aspects, a kit may comprise a notice in the form prescribed by a government agency regulating the manufacture, use or sale of pharmaceutical products, which notice reflects approval by the agency of manufacture, use of sale for human administration. In other embodiments, a kit may further comprise a virtual package. As used herein, a “virtual package” refers to components of a kit that are associated by directions on one or more physical or virtual kit components instructing the user how to obtain the other components. A non-limiting example of a virtual package includes a bag or other container containing one component and directions instructing a subject to go to a website, contact a recorded message or a fax-back service, view a visual message, or contact a caregiver or instructor to obtain instructions on how to use the kit or safety or technical information about one or more components of a kit.

In other embodiments, a kit may be a single package. As used herein, the term “single package” means that the components of a kit are physically associated in or with one or more containers and considered a unit for manufacture, distribution, sale, or use. Examples of containers include, but are not limited to, bags, boxes, cartons, bottles, packages such as shrink-wrap packages, stapled or otherwise affixed components, or combinations thereof. In some embodiments, a kit may comprise one or more components to assist with ex vivo immune cell expansion as described herein. In some aspects, such components may include culture flasks, antibiotics for culture medium, serum for culture medium, a control cell line, reagents for detection of immune cell markers, or a combination thereof.

### (III) Methods

Aspects of the present disclosure include methods of ex vivo expansion of immune cells as disclosed herein. Other aspects of the present disclosure include methods of administering immune cell therapy compositions disclosed herein to a subject in need thereof.

### (a) Methods of Ex Vivo Expansion of Immune Cells

In some embodiments, methods preparing an immunotherapy composition disclosed herein can encompass immune cells isolated from a subject. In some embodiments, methods herein may include preparation of an initial immune cell population. In some aspects, an initial immune cell population can be prepared from peripheral blood, umbilical cord blood, and/or bone marrow. In other aspects, an initial immune cell population can be prepared from peripheral blood mononuclear cells (PBMCs). In still other aspects, an initial immune cell population can be prepared from a leukapheresis sample. In yet other aspects, an initial immune cell population can be prepared from tumor-infiltrated lymphocytes, tissue-infiltrated lymphocytes, lymph nodes, thymus, and/or secondary lymphoid organs. In some embodiments, an initial immune cell population for use in methods disclosed herein may be categorized as lymphocytes, neutrophils, granulocytes, mast cells, monocytes/macrophages, and/or dendritic cells (DCs). In some aspects, an initial immune cell population for use in methods disclosed herein may be least one lymphocyte. In some aspects, an initial immune cell population for use in methods disclosed herein may be a lymphocyte. In some aspects, an initial immune cell population for use in methods disclosed herein may be a natural killer (NK) cell, a CD4 T cell, or a CD8 T cell. In some aspects, an initial immune cell population for use in methods disclosed herein may be a DC cell.

In some embodiments, an initial immune cell population may be separated by phenotypic selection for markers including but not limited to CD3, CD28, CD3/CD28, and/or CD14 selection. In some embodiments, DC cells and/or T cells can be isolated from an initial immune cell population as disclosed herein. In some embodiments, DC cells can be isolated from an initial immune cell population as measured by CD11c expression on their surface. In some aspects, DCs can be monocyte-derived dendritic cells (DCs). In some embodiments, T cells can be isolated from an initial immune cell population as measured by CD3 expression on their surface.

In some embodiments, methods preparing an immunotherapy composition disclosed. herein can encompass obtaining at least one target cancer cell. In some aspects, target cells for use in the methods herein can be tumor cells. In some aspects, target cells for use in the methods herein can be obtained from surgery, biopsy, cell banks, commercial vendors, repositories, or healthy donors. In some embodiments, target cancer cells and/or the aforementioned immune cells used preparing an initial immune cell population can be obtained from patients, healthy donors, blood banks, cell banks, commercial vendors, and/or repositories. In some embodiments, autologous immune cells are used. In some embodiments, immune cells and target cells for use in the methods disclosed herein can be unmatched or with unknown HLA status. In some embodiments, immune cells and target cells for use in the methods disclosed herein can be matched on at least 1 HLA to target cells.

In some embodiments, methods preparing an immunotherapy composition disclosed herein can encompass exposing at least one target cancer cell to at least one PBNP composition disclosed herein. In some aspects, at least one target cancer cell can be exposed to at least one PBNP composition disclosed herein, wherein the PBNP is uncoated. In some aspects, at least one target cancer cell can be exposed to at least one PBNP composition disclosed herein, wherein the PBNP is coated. In some aspects, at least one target cancer cell can be exposed to at least one PBNP composition disclosed herein, wherein the PBNP is coated with one or more immune signals.

In some embodiments, methods preparing an immunotherapy composition disclosed herein can encompass subjecting the at least one target cancer cell and the at least one PBNP to photothermal therapy (PTT) (i.e., PBNP-PTT). As a result of administering PTT according to the methods disclosed herein, when the PBNPs are irradiated with incident light (typically a monochromatic light source such as a laser) they heat up by a process known as photothermal conversion wherein the resultant temperatures can range from ambient temperature to around 150° C. based on the conditions used for photothermal heating. Photothermal heating via incident light and PBNPs is termed PBNP-PTT. In some embodiments, methods preparing an immunotherapy composition disclosed herein can encompass subjecting the at least one target cancer cell to PBNP-PTT. In accordance with these embodiments, PBNP-PTT as used herein can generate a thermal dose ranging from about 1 to about 25 log (Σcumulative equivalent minutes at 43° C. (CEM43)). In some embodiments, PBNP-PTT can be administered to the target cancer cells herein at a thermal dose that can generate target cell immunogenicity.

In some embodiments, methods preparing an immunotherapy composition disclosed herein can encompass co-culturing isolated DCs with target cells. In some aspects, target cells can be used in methods of co-culturing herein directly after PBNP-PTT. In some aspects, target cells can be used in methods of co-culturing herein after culturing in serum-rich or serum-free media for about 1 to about 120 hours post-PBNP-PTT. In some aspects, target cells can be first subjected to PBNP-PTT, lysed after PBNP-PTT, and the target cell lysate can be applied to the isolated DCs. Non-limiting examples of methods suitable for lysing the target cells after PBNP-PTT can include mechanical disruption, liquid homogenization, high frequency sound waves, freeze/thaw cycles, manual grinding.

In some embodiments, methods preparing an immunotherapy composition disclosed herein can encompass co-culturing target cells treated with PBNP-PTT as described herein with dendritic cells, T cells, PBMCs, or any combination thereof. In some aspects, target cells treated with PBNP-PTT can be co-cultured in a medium comprising T cells matched on at least 1 HLA. In some aspects, target cells treated with PBNP-PTT can be co-cultured in a medium comprising at least one cytokine and/or antibodiy. Examples of cytokines can include, but are but not limited to GM-CSF, IL-4, TNFa, IFNy, IL1B, IL-6, LPS, IL-7, IL-12, IL-15, IL-2. Examples of antibodies can include, but not limited to, anti-CD3, anti-CD28, anti-CD137, anti-CTLA-4, anti-PD-1. In some embodiments, methods preparing an immunotherapy composition disclosed herein can encompass co-culturing target cells for a period of time ranging from about 5 days to about 35 days or about 6 days to about 30 days. In some embodiments, methods preparing an immunotherapy composition disclosed herein can encompass co-culturing target cells treated with PBNP-PTT as described herein in a medium comprising T cells isolated from the initial immune population.

In some embodiments, methods preparing an immunotherapy composition disclosed herein can encompass harvesting immune cells specific to at least one target cancer cell from the expanded co-culture. In some embodiments, methods preparing an immunotherapy composition disclosed herein can encompass harvesting T cells specific to at least one target cancer cell from the expanded co-culture. In some aspects, T cells harvested from the expanded co-culture can express one or more markers of T cell activation. in some examples, markers of T cell activation to be measured herein can include CD45RO, CD137, CD25, CD279, CD179, CD62L, HLA-DR, CD69, CD223 (LAG3), CD134 (0X40), CD183 (CXCR3), CD27 (IL-7Ra), CD366 (TIM3), CD80, CD152 (CTLA-4), CD28, CD278 (ICOS), CD154 (CD40L), or any combination thereof.

In some embodiments, methods of preparing an immunotherapy composition disclosed herein can result in a population of T cells having improved specificity to tumor cells compared to native. In some embodiments, methods of preparing an immunotherapy composition disclosed herein can result in a population of T cells having improved specificity to tumor cells having the same phenotype as the target cells used in the methods herein compared to native T cells. In some embodiments, methods of preparing an immunotherapy composition disclosed herein can result in a population of T cells having about 1% more to about 100% more (e.g., about 1%, about 5%, about 10%, about 20%, about 30%, about 40%, about 50%, about 60%, about 70%, about 80%, about 90%, about 99%, about 100% more) specificity to tumor cells having the same phenotype as the target cells used in the methods herein compared to native T cells. In some embodiments, methods of preparing an immunotherapy composition disclosed herein can result in a population of T cells having improved specificity to GBM tumor cells compared to native T cells. In some embodiments, methods of preparing an immunotherapy composition disclosed herein can result in a population of T cells having improved specificity to breast cancer tumor cells compared to native T cells.

### (b) Methods of Use

In certain embodiments, immunotherapy compositions disclosed herein can be administered to a subject according to an intravenous, intraperitoneal, intradermal, subcutaneous, intrathecal, intracerebral, peri-tumoral, and/or intra-tumoral route of administration. In some embodiments, immunotherapy compositions disclosed herein may be administered by parenteral administration. As used herein, “by parenteral administration” refers to administration of immune cell therapy compositions disclosed herein via a route other than through the digestive tract. In some embodiments, immune cell therapy compositions disclosed herein may be administered by parenteral injection. In some aspects, administration of the disclosed immune cell therapy compositions by parenteral injection may be by subcutaneous, intramuscular, intravenous, intraperitoneal, intracardiac, intraarticular, or intracavernous injection. In other aspects, administration of the disclosed immune cell therapy compositions by parenteral injection may be by slow or bolus methods as known in the field. In some embodiments, the route of administration by parenteral injection can be determined by the target location. In some aspects, compositions disclosed herein may be administered to a solid tumor.

In various embodiments, the dose of immune cell therapy compositions disclosed herein to be administered are not particularly limited, and may be appropriately chosen depending on conditions such as a purpose of preventive and/or therapeutic treatment, a type of a disease, the body weight or age of a subject, severity of a disease and the like. In other embodiments, administration of a dose of an immune cell therapy composition disclosed herein may comprise an effective amount of the composition disclosed herein. As used herein, the term “effective amount” refers to an amount of administered composition that treats an infectious disease, an autoimmune disease, an immune deficiency disease, a cancer, graft-versus-host disease (GVHD), transplant intolerance, or a combination thereof.

An effective amount of an immune cell therapy composition disclosed herein to be delivered to a subject may be an amount that does not result in undesirable systemic side effects. In various embodiments, immune cell therapy compositions administered as disclosed herein may comprise about 5% to about 95%, about 15% to about 85%, or about 25% to about 75% total genetically modified immune cells by total weight of the composition. In other embodiments, immune cell therapy compositions administered as disclosed herein may comprise about 5% to about 95%, about 15% to about 85%, or about 25% to about 75% total genetically modified immune cells with increased cytolytic activity by total weight of the composition. In still other embodiments, immune cell therapy compositions administered as disclosed herein may comprise about 5% to about 95%, about 15% to about 85%, or about 25% to about 75% total genetically modified immune cells with increase cytolytic gene expression by total weight of the composition.

In some embodiments, an immune cell therapy composition disclosed herein may be administered to a subject in need thereof once. In some embodiments, an immune cell therapy composition disclosed herein may be administered to a subject in need thereof more than once. In other embodiments, a first administration of an immune cell therapy composition disclosed herein may be followed by a second administration of an immune cell therapy composition disclosed herein. In some embodiments, a first administration of an immune cell therapy composition disclosed herein may be followed by a second and third administration of an immune cell therapy composition disclosed herein. In some embodiments, a first administration of an immune cell therapy composition disclosed herein may be followed by a second, third, and fourth administration of an immune cell therapy composition disclosed herein. In some embodiments, a first administration of an immune cell therapy composition disclosed herein may be followed by a second, third, fourth, and fifth administration of an immune cell therapy composition disclosed herein.

The number of times a immunotherapy compositions disclosed herein may be administered to a subject in need thereof can depend on the discretion of a medical professional, the severity of the disease, and the subject's response to the formulation. In some embodiments, an immune cell therapy composition disclosed herein may be administered continuously; alternatively, the dose of immune cell therapy composition being administered may be temporarily reduced or temporarily suspended for a certain length of time (i.e., a “drug holiday”). In some aspects, the length of the drug holiday can vary between 2 days and 1 year, including by way of example only, 2 days, 1 week, 1 month, 6 months, and 1 year. In another aspect, dose reduction during a drug holiday may be from 10%-100%, including by way of example only 10%, 25%, 50%, 75%, and 100%.

In various embodiments, the desired daily dose of immune cell therapy compositions disclosed herein may be presented in a single dose or as divided doses administered simultaneously (or over a short period of time) or at appropriate intervals. In other embodiments, administration of an immune cell therapy composition disclosed herein may be administered to a subject about once a day, about twice a day, about three times a day. In still other embodiments, administration of an immune cell therapy composition disclosed herein may be administered to a subject at least once a day, at least once a day for about 2 days, at least once a day for about 3 days, at least once a day for about 4 days, at least once a day for about 5 days, at least once a day for about 6 days, at least once a day for about 1 week, at least once a day for about 2 weeks, at least once a day for about 3 weeks, at least once a day for about 4 weeks, at least once a day for about 8 weeks, at least once a day for about 12 weeks, at least once a day for about 16 weeks, at least once a day for about 24 weeks, at least once a day for about 52 weeks and thereafter. In a preferred embodiment, administration of an immune cell therapy composition disclosed herein may be administered to a subject once about 4 weeks.

In some embodiments, an immune cell therapy composition as disclosed may be initially administered followed by a subsequent administration of one for more different compositions or treatment regimens. In other embodiments, an immune cell therapy composition as disclosed may be administered after administration of one for more different compositions or treatment regimens. In some aspects, different compositions may be cytokines.

In certain embodiments, the present disclosure provides methods for treating cancer in a subject herein. In some embodiments, methods herein may be used for treating a solid tumor in a subject. Non-limiting examples of solid tumors that may be treated by the methods herein may include pancreatic ductal adenocarcinoma (PDA), colorectal cancer (CRC), melanoma, cholangiocarcinoma, breast cancer, small cell and non-small cell lung cancer, upper and lower gastrointestinal malignancies, gastric cancer, squamous cell head and neck cancer, genitourinary cancer, hepatocellular carcinoma, ovarian cancer, sarcomas, mesothelioma, glioblastoma, esophageal cancer, bladder cancer, urothelial cancer, renal cancer, cervical and/or endometrial cancer. In some embodiments, the cancer that may be treated by the methods herein may be adrenal cancer, adrenocortical carcinoma, anal cancer, appendix cancer, bile duct cancer, bladder cancer, bone cancer (e.g., Ewing sarcoma tumors, osteosarcoma, malignant fibrous histiocytoma), brain cancer (e.g., astrocytomas, brain stem glioma, craniopharyngioma, ependymoma), bronchial tumors, cholangiocarcinoma, cholangiosarcoma, central nervous system tumors, breast cancer, Castleman disease, cervical cancer, colon cancer, rectal cancer, colorectal cancer, endometrial cancer, esophageal cancer, eye cancer, gallbladder cancer, gastrointestinal cancer, gastrointestinal carcinoid tumors, gastrointestinal stromal tumors, genitourinary cancers, gestational trophoblastic disease, heart cancer, Kaposi sarcoma, kidney cancer, laryngeal cancer, hypopharyngeal cancer, leukemia (e.g., acute lymphoblastic leukemia, acute myeloid leukemia, chronic lymphocytic leukemia, chronic myelogenous leukemia), liver cancer, lung cancer (for example, non-small cell lung cancer, NSCLC, and small cell lung cancer, SCLC), lymphoma (e.g., AIDS-related lymphoma, Burkitt lymphoma, cutaneous T cell lymphoma, Hogkin lymphoma, Non-Hogkin lymphoma, primary central nervous system lymphoma), malignant mesothelioma, multiple myeloma, myelodysplastic syndrome, nasal cavity cancer, paranasal sinus cancer, pancreatic duct adenocarcinoma (PDA) nasopharyngeal cancer, neuroblastoma, oral cavity cancer, oropharyngeal cancer, osteosarcoma, ovarian cancer, pancreatic cancer, penile cancer, pituitary tumors, prostate cancer, retinoblastoma, rhabdomyosarcoma, rhabdoid tumor, salivary gland cancer, sarcoma, skin cancer (e.g., basal cell carcinoma, melanoma), squamous cell head and neck cancer, small intestine cancer, stomach cancer, teratoid tumor, testicular cancer, throat cancer, thymus cancer, thyroid cancer, unusual childhood cancers, upper and lower gastrointestinal malignancies (including, but not limited to, esophageal, gastric, and hepatobiliary cancer), urethral cancer, uterine cancer, uterine sarcoma, vaginal cancer, vulvar cancer, Waldenstrom macroglobulinemia, and Wilms tumor. In some embodiments, the cancer may be selected from hematological malignancies including acute lymphoblastic leukemia, chronic lymphocytic leukemia, lymphomas, multiple myeloma, acute myelogenous leukemia, chronic myelogenous leukemia, myelodysplastic syndromes and the myeloproliferative neoplasms, such as essential thrombocythemia, polycythemia vera, myelofibrosis, gallbladder cancer (adenocarcinomas or squamous cell carcinoma), or any combination thereof. In some embodiments, the symptom(s) associated with the disease (e.g., cancer) may include, but are not limited to, anemia, loss of appetite, irritation of bladder lining, bleeding and bruising (thrombocytopenia), changes in taste or smell, constipation, diarrhea, dry mouth, dysphagia, edema, fatigue, hair loss (alopecia), infection, infertility, lymphedema, mouth sores, nausea, pain, peripheral neuropathy, tooth decay, urinary tract infections, problems with memory and concentration, or any combination thereof.

A subject having any of the above noted cancers can be identified by routine medical examination, e.g., laboratory tests, organ functional tests, genetic tests, interventional procedure (biopsy, surgery) and/or any relevant imaging modalities. In some embodiments, a subject to be treated by methods described herein may be a human cancer patient who has undergone or is subjecting to an anti-cancer therapy, for example, chemotherapy, radiotherapy, immunotherapy, or surgery. In some embodiments, subjects may have received prior immunomodulatory anti-tumor agents. Non-limiting examples of such immunomodulatory agents include, but are not limited to as anti -PD1, anti-PD-L1, anti-CTLA-4, anti-OX40, anti-CD137, etc. In some embodiments, a subject herein can show disease progression through the treatment. In other embodiments, a subject herein may be resistant to the treatment (either de novo or acquired). In some embodiments, such a subject may demonstrate as having one or more advanced malignancies (e.g., inoperable or metastatic). Alternatively or in addition, in some embodiments, a subject herein may have no standard therapeutic options available or ineligible for standard treatment options, which refer to therapies commonly used in clinical settings for treating the corresponding solid tumor (i.e., a subject having a terminal cancer). Alternatively or in addition, in some embodiments, a subject herein may be a human patient having a refractory disease. As used herein, “refractory” refers to cancer and/or tumor that does not respond to and/or becomes resistant to a treatment. In some instances, a subject herein may be a human patient having a relapsed disease. As used herein, “relapsed” or “relapses” refers to a tumor that returns or progresses following a period of improvement (e.g., a partial or complete response) with treatment.

In some embodiments, the disclosure provides a method for treating cancer in a subject, the method comprising administering to a subject in need thereof an effective amount an immunotherapy compositions disclosed herein. As used herein, “an effective amount” refers to the amount of each active agent required to confer therapeutic effect on the subject, either alone or in combination with one or more other active agents. Determination of whether an amount of immunotherapy achieved the therapeutic effect would be evident to one of skill in the art. Effective amounts vary, as recognized by those skilled in the art, depending on the particular condition being treated, the severity of the condition, the individual patient parameters including age, physical condition, size, gender and weight, the duration of the treatment, the nature of concurrent therapy (if any), the specific route of administration and like factors within the knowledge and expertise of the health practitioner. These factors are well known to those of ordinary skill in the art and can be addressed with no more than routine experimentation. It is generally preferred that a maximum dose of the individual components or combinations thereof be used, that is, the highest safe dose according to sound medical judgment.

In some embodiments, a subject treated with any of the methods herein can have completed an additional therapeutic regimen, be receiving an additional therapeutic regimen, or can receive an additional therapeutic regimen following treatment according to the methods herein. In some embodiments, an additional therapeutic regimen for use herein can include administering a chemotherapeutic agent. In some embodiments, a chemotherapeutic agent can be a cell cycle inhibitor. As used herein “cell cycle inhibitor” can include a chemotherapeutic agent that inhibits or prevents the division and/or replication of cells. In some embodiments, a cell cycle inhibitor can include a chemotherapeutic agent such as doxorubicin, melphlan, roscovitine, mitomycin C, hydroxyurea, 5-fluorouracil, cisplatin, ara-C, etoposide, gemcitabine, bortezomib, sunitinib, sorafenib, sodium valproate, a HDAC inhibitor, or dacarbazine. More examples of additional chemotherapeutic agents include but are not limited to HDAC inhibitors such as FR01228, trichostatin A, SAHA and/or PDX101. In some embodiments, the cell cycle inhibitor is a DNA synthesis inhibitor. As used herein, a “DNA synthesis inhibitor” can include a chemotherapeutic agent that inhibits or prevents the synthesis of DNA by a cancer cell. Examples of DNA synthesis inhibitors include but are not limited to AraC (cytarabine), 6-mercaptopurine, 6-thioguanine, 5-fluorouracil, capecitabine, floxuridine, gemcitabine, decitabine, vidaza, fludarabine, nelarabine, cladribine, clofarabine, pentostatin, thiarabine, troxacitabine, sapacitabine or forodesine. More examples of additional chemotherapeutic agents include, but are not limited to, FLT3 inhibitors such as Semexanib (SCT5416), Sunitinib (SU 11248), Midostaurin (PKC412), Lestautinib (CEP-701), Tandutinib (MLN518), CHIR-258, Sorafenib (BAY-43-9006) and/or KW-2449. More non-limiting examples of additional chemotherapeutic agents include farnesyltransferase inhibitors such as tipifarnib (R1 15777, Zarnestra), lonafarnib (SCH66336, Sarasar™) and/or BMS-214662. More examples of additional chemotherapeutic agents include, but are not limited to, topoisomerase II inhibitors such as the epipodophyllotoxins etoposide, teniposide, anthracyclines doxorubicin and/or 4-epi-doxorubicin. More non-limiting examples of additional chemotherapeutic agents include P-glycoprotein modulators such as zosuquidar trihydrochloride (Z3HCL), vanadate, or verapamil. More non-limiting examples of additional chemotherapeutic agents include hypomethylating agents such as 5-aza-cytidine or 2′ deoxyazacitidine.

In some embodiments, an additional therapeutic regimen for use herein can include administering one or more immunomodulatory agents. Non-limiting examples of such immunomodulatory agents can include on or more monoclonal antibody (mAb) therapies. In some embodiments, a mAb therapy can target HER2; EGFR; VEGFR; VEGF; CD-20; CD-22; CD-52; CD-33; CD-30; CD19/CD3; CD38; CTLA-4; PD-1; PD-L1; RANKL; GD2; PDGFR; SLAMF7, or any combination thereof. Non-limiting examples of mAb therapies suitable for use herein can include adotrastuzumab, trastuzumab, pertuzumab, cetuximab, panitumumab, necitumumab, ramucirumab, bevacizumab, rituximab, ofatumumab, ibritumomab, tositumomab, obinutuzumab, inotuzumab, alemtuzumab, gemtuzumab, brentuximab, blinatumomab, daratumumab, ipilimumab, nivolumab, atezolizumab, avelumab, cemiplimab, pembrolizumab, durvalumab, denosumab, dinutuximab, olaratumab, elotuzumab, and the like.

In some embodiments, an additional therapeutic regimen for use herein can include administering one or more small molecules. Non-limiting examples of such small molecules can include imatinib, dasatinib, nilotinib, bosutinib, regorafenib, ponatinib, sunitinib, sorafenib, erdafitinib, lenvatinib, pazopanib, afatinib, gefitinib, osimertinib, vandetanib, erlotinib, lapatinib, dacomitinib, neratinib, ribociclib, abemaciclib, palbociclib, cabozantinib, crizotinib, axitinib, alectinib, vemurafenib, encorafenib, dabrafenib, olaparib, rucaparib, talazoparib, niraparib, larotrectinib, entrectinib, lorlatinib, ibrutinib, cobimetinib, binimetinib, trametinib, brigatinib, cgilteritinib, ceritinib, ivosidenib, carfilzomib, marizomib, alpelisib, duvelisib, copanlisib, and the like.

In some embodiments, treatment of a subject with a precision cancer therapy (e.g., immunotherapy) after determining the molecular markers as disclosed herein, may prevent cancer progression. In some embodiments, treatment of a subject after determining the molecular markers as disclosed herein, may ameliorate one or more symptoms associated with cancer. In still other aspects, treatment of a subject with a precision cancer therapy (e.g., immunotherapy) after determining the molecular markers as disclosed herein, may reduce risk of cancer recurrence in the subject. In other aspects, treatment of a subject with a precision cancer therapy (e.g., immunotherapy) after determining the molecular markers as disclosed herein, may slow tumor growth in the subject. In some embodiments, treatment of a subject with a precision cancer therapy (e.g., immunotherapy) after determining the molecular markers as disclosed herein, may reduce the risk of metastasis in the subject.

In certain embodiments, compositions (e.g., immunotherapy compositions) disclosed herein can treat and/or prevent cancer in a subject in need wherein the subject has a molecular markers score over the threshold as determined herein. In some embodiments, methods of treatment disclosed herein (e.g., immunotherapy) can impair tumor growth compared to tumor growth in an untreated subject with identical disease condition and predicted outcome. In some embodiments, tumor growth can be stopped following treatments (e.g., immunotherapy) according to the methods disclosed herein. In other embodiments, tumor growth can be impaired at least about 5% or greater to at least about 100%, at least about 10% or greater to at least about 95% or greater, at least about 20% or greater to at least about 80% or greater, at least about 40% or greater to at least about 60% or greater compared to an untreated subject with identical disease condition and predicted outcome. In other words, tumors in a subject treated according to the methods disclosed herein may grow at least 5% less (or more as described above) when compared to an untreated subject with identical disease condition and predicted outcome. In some embodiments, tumor growth can be impaired at least about 5% or greater, at least about 10% or greater, at least about 15% or greater, at least about 20% or greater, at least about 25% or greater, at least about 30% or greater, at least about 35% or greater, at least about 40% or greater, at least about 45% or greater, at least about 50% or greater, at least about 55% or greater, at least about 60% or greater, at least about 65% or greater, at least about 70% or greater, at least about 75% or greater, at least about 80% or greater, at least about 85% or greater, at least about 90% or greater, at least about 95% or greater, at least about 100% compared to an untreated subject with identical disease condition and predicted outcome. In some embodiments, tumor growth can be impaired at least about 5% or greater to at least about 10% or greater, at least about 10% or greater to at least about 15% or greater, at least about 15% or greater to at least about 20% or greater, at least about 20% or greater to at least about 25% or greater, at least about 25% or greater to at least about 30% or greater, at least about 30% or greater to at least about 35% or greater, at least about 35% or greater to at least about 40% or greater, at least about 40% or greater to at least about 45% or greater, at least about 45% or greater to at least about 50% or greater, at least about 50% or greater to at least about 55% or greater, at least about 55% or greater to at least about 60% or greater, at least about 60% or greater to at least about 65% or greater, at least about 65% or greater to at least about 70% or greater, at least about 70% or greater to at least about 75% or greater, at least about 75% or greater to at least about 80% or greater, at least about 80% or greater to at least about 85% or greater, at least about 85% or greater to at least about 90% or greater, at least about 90% or greater to at least about 95% or greater, at least about 95% or greater to at least about 100% compared to an untreated subject with identical disease condition and predicted outcome.

In some embodiments, treatment of tumors according to the methods disclosed herein (e.g., immunotherapy) can result in a shrinking of a tumor in comparison to the starting size of the tumor. In some embodiments, tumor shrinking is at least about 5% or greater to at least about 10% or greater, at least about 10% or greater to at least about 15% or greater, at least about 15% or greater to at least about 20% or greater, at least about 20% or greater to at least about 25% or greater, at least about 25% or greater to at least about 30% or greater, at least about 30% or greater to at least about 35% or greater, at least about 35% or greater to at least about 40% or greater, at least about 40% or greater to at least about 45% or greater, at least about 45% or greater to at least about 50% or greater, at least about 50% or greater to at least about 55% or greater, at least about 55% or greater to at least about 60% or greater, at least about 60% or greater to at least about 65% or greater, at least about 65% or greater to at least about 70% or greater, at least about 70% or greater to at least about 75% or greater, at least about 75% or greater to at least about 80% or greater, at least about 80% or greater to at least about 85% or greater, at least about 85% or greater to at least about 90% or greater, at least about 90% or greater to at least about 95% or greater, at least about 95% or greater to at least about 100% (meaning that the tumor is completely gone after treatment) compared to the starting size of the tumor.

In some embodiments, treatments administered according to the methods disclosed herein (e.g., immunotherapy) can improve patient life expectancy compared to the cancer life expectancy of an untreated subject with identical disease condition and predicted outcome. As used herein, “patient life expectancy” is defined as the time at which 50 percent of subjects are alive and 50 percent have passed away. In some embodiments, patient life expectancy can be indefinite following treatment according to the methods disclosed herein. In other aspects, patient life expectancy can be increased at least about 5% or greater to at least about 100%, at least about 10% or greater to at least about 95% or greater, at least about 20% or greater to at least about 80% or greater, at least about 40% or greater to at least about 60% or greater compared to an untreated subject with identical disease condition and predicted outcome. In some embodiments, patient life expectancy can be increased at least about 5% or greater, at least about 10% or greater, at least about 15% or greater, at least about 20% or greater, at least about 25% or greater, at least about 30% or greater, at least about 35% or greater, at least about 40% or greater, at least about 45% or greater, at least about 50% or greater, at least about 55% or greater, at least about 60% or greater, at least about 65% or greater, at least about 70% or greater, at least about 75% or greater, at least about 80% or greater, at least about 85% or greater, at least about 90% or greater, at least about 95% or greater, at least about 100% compared to an untreated subject with identical disease condition and predicted outcome. In some embodiments, patient life expectancy can be increased at least about 5% or greater to at least about 10% or greater, at least about 10% or greater to at least about 15% or greater, at least about 15% or greater to at least about 20% or greater, at least about 20% or greater to at least about 25% or greater, at least about 25% or greater to at least about 30% or greater, at least about 30% or greater to at least about 35% or greater, at least about 35% or greater to at least about 40% or greater, at least about 40% or greater to at least about 45% or greater, at least about 45% or greater to at least about 50% or greater, at least about 50% or greater to at least about 55% or greater, at least about 55% or greater to at least about 60% or greater, at least about 60% or greater to at least about 65% or greater, at least about 65% or greater to at least about 70% or greater, at least about 70% or greater to at least about 75% or greater, at least about 75% or greater to at least about 80% or greater, at least about 80% or greater to at least about 85% or greater, at least about 85% or greater to at least about 90% or greater, at least about 90% or greater to at least about 95% or greater, at least about 95% or greater to at least about 100% compared to an untreated patient with identical disease condition and predicted outcome.

In some embodiments, a subject to be treated by any of the methods herein (e.g., immunotherapy) can present with one or more cancerous solid tumors, metastatic nodes, of a combination thereof. In some embodiments, a subject herein can have a cancerous tumor cell source that can be less than about 0.2 cm³ to at least about 20 cm³ or greater, at least about 2 cm³ to at least about 18 cm³ or greater, at least about 3 cm³ to at least about 15 cm³ or greater, at least about 4 cm³ to at least about 12 cm³ or greater, at least about 5 cm³ to at least about 10 cm³ or greater, or at least about 6 cm³ to at least about 8 cm³ or greater.

In some embodiments, any of the methods disclosed herein (e.g., immunotherapy) can further include monitoring occurrence of one or more adverse effects in the subject. Exemplary adverse effects include, but are not limited to, hepatic impairment, hematologic toxicity, neurologic toxicity, cutaneous toxicity, gastrointestinal toxicity, or a combination thereof. When one or more adverse effects are observed, the method disclosed herein can further include reducing or increasing the dose of one or more of the immunotherapy, the dose of one or more anticancer drugs (e.g., chemotherapeutics, small molecules, mAbs) or both depending on the adverse effect or effects in the subject.

### EXAMPLES

The following examples are included to illustrate certain embodiments. It should be appreciated by those of skill in the art that the techniques disclosed in the examples which follow represent techniques discovered to function well in the practice of the claimed methods, compositions and apparatus. However, those of skill in the art should, in light of the present disclosure, appreciate that changes can be made in some embodiments which are disclosed and still obtain a like or similar result without departing from the spirit and scope of the invention.

### Example 1

In an exemplary method, glioblastoma-specific T cells were generated using PBNPs compositions. In brief, glioblastoma (GBM)-specific T cells were generated using compositions containing PBNPs by following the manufacturing methods of the present application. In one example, U87 cells (a human primary glioblastoma cell line) were used as target cells and a composition containing PBNPs or a combination of PBNPs and immune signals was administered to U87 cells at an optimal thermal dose. The optimal thermal dose was optimized and measured by thermal dose and immunogenic biochemical correlates, e.g., by measuring levels of immunogenic biochemical correlates at different thermal doses. PBNPs were irradiated with incident light or a monochromatic light source, such as a laser, having a wavelength within the range of the absorbance peak of PBNPs to generate thermal conversion of PBNPs through photothermal heating. A PBNP-PTT process was applied to disrupt, kill, or break U87 cells by utilizing the photothermal conversion of PBNPs. Subsequently, PBNP-PTT treated U87 cells were co-cultured with immune cells, including dendritic cells and/or T cells. The manufactured antigen-specific immune cells were confirmed to be specific to U87 cells using IFN-y ELISpot. The manufactured antigen-specific immune cells were expanded and characterized for phenotype and U87-specific cytotoxicity.

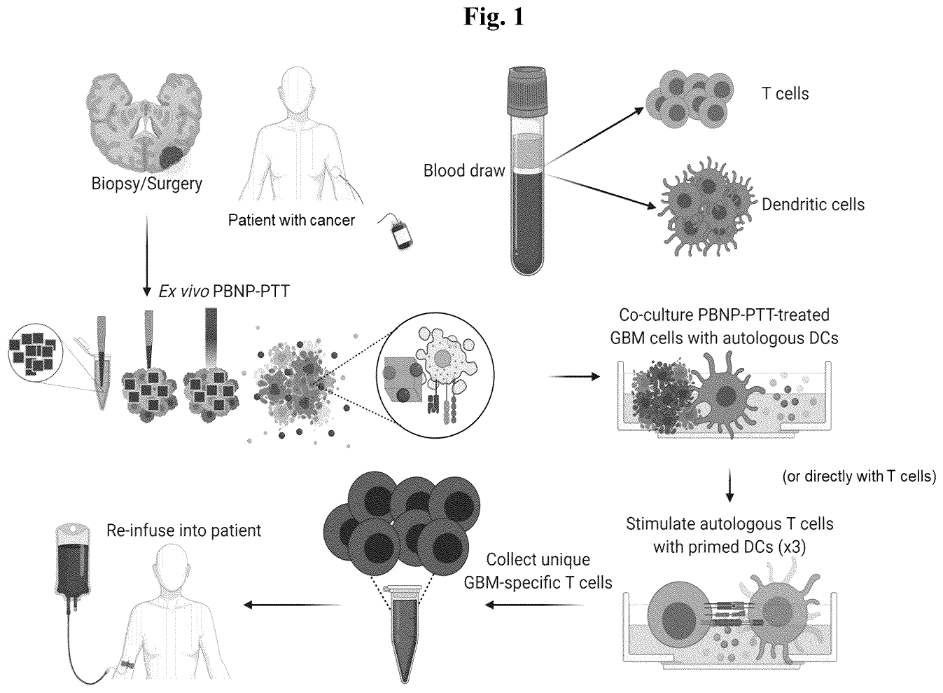

FIG. 1 shows an exemplary treatment scheme. A tumor biopsy containing GBM cells was obtained from the cancer patient. A composition containing PBNPs was administered to GBM cells at an optimal thermal dose using incident light or a monochromatic light source. A PBNP-PTT process was applied to disrupt, kill, or break GBM cells. Subsequently, PBNP-PTT treated GBM cells were co-cultured with autologous dendritic cells and/or directly with T cells to stimulate autologous T cells with primed dendritic cells. The GBM-specific T cells were collected, characterized, and expanded. The manufactured GBM-specific T cells were re-infused into the cancer patient.

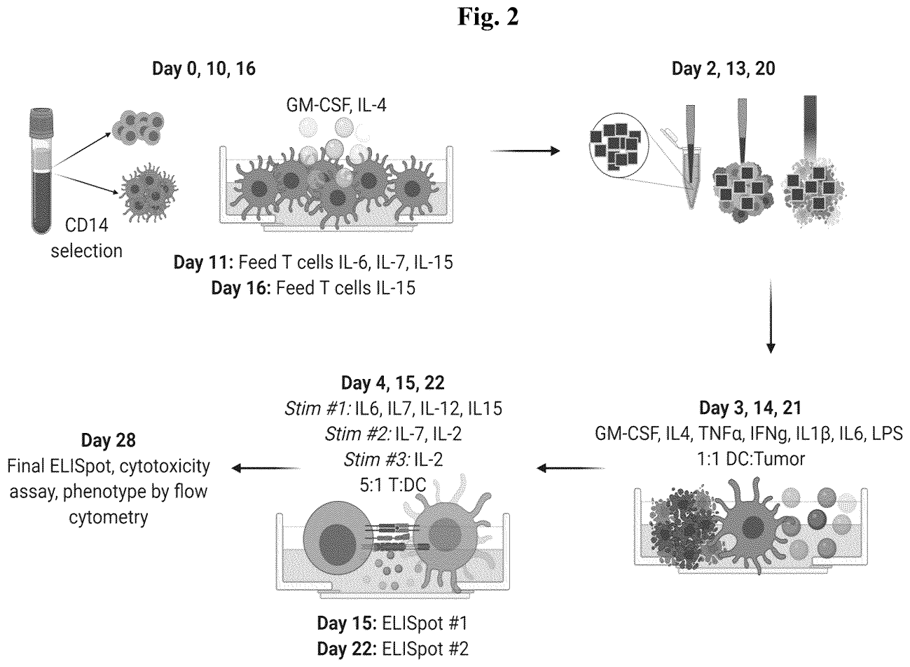

FIG. 2 shows an exemplary first manufacture protocol. T cells and dendritic cells were isolated using CD14 selection (Day 0, 10, and 16). Isolated T cells and dendritic cells were cultured in the presence of GM-CSF and IL-4. (Day 11: feed T cells, IL-6, IL-7, IL-15; Day 16: feed T cells, IL-15) A composition containing PBNPs was administered to target cells at an optimal thermal dose using incident light or a monochromatic light source. A PBNP-PTT process was applied to disrupt, kill, or break target cells. (Day 2. 13, 20) Subsequently, PBNP-PTT treated target cells were co-cultured with dendritic cells and/or directly with T cells to stimulate autologous T cells with primed dendritic cells (DC). (Day 3, 14, 21, GM-CSF, IL4, TNFα, IFNg, IL1β, IL6, LPS, the ratio of DC to the tumor is 1:1) (Day 4, 15, 22; Stim #1: IL6, IL7, IL12, IL15; Stim #2: IL7, IL2; Stim #3: IL-2, the ratio of T cell to DC is 5:1) (Day 15: ELISpot #1, Day 22: ELISpot #2). The manufactured antigen-specific immune cells were confirmed to be specific to target cells using final ELISpot. The manufactured antigen-specific immune cells were expanded and characterized for phenotype and cytotoxicity using flow cytometry.

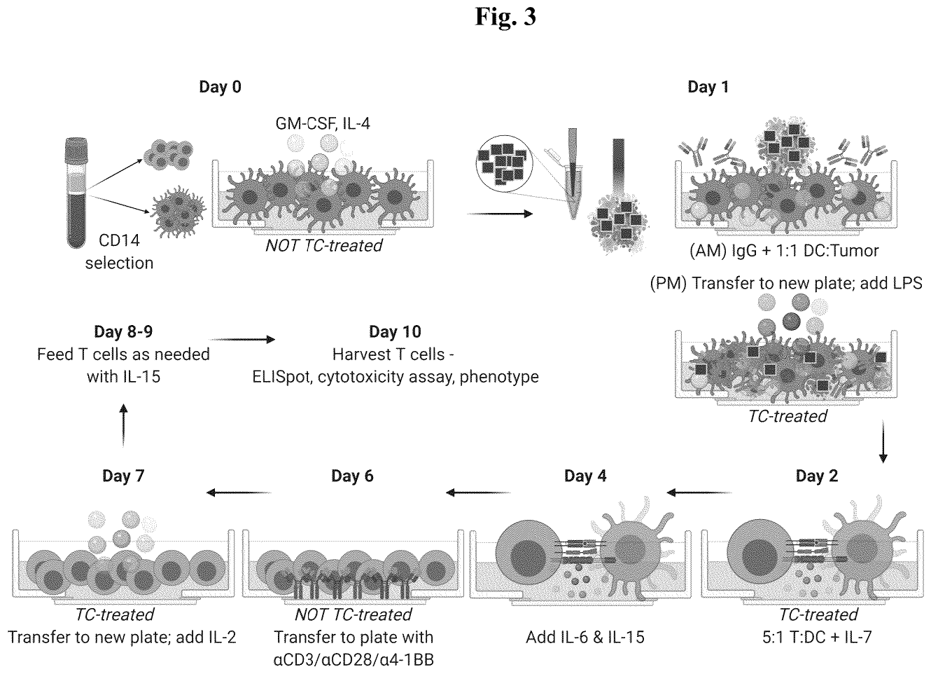

FIG. 3 shows an exemplary second manufacture protocol. T cells and dendritic cells were isolated using CD14 selection (Day 0). Isolated T cells and dendritic cells were cultured in the presence of GM-CSF and IL-4 (not TC-treated). A composition containing PBNPs was administered to target cells at an optimal thermal dose using incident light or a monochromatic light source. A PBNP-PTT process was applied to disrupt, kill, or break target cells. Subsequently, PBNP-PTT treated target cells were co-cultured with dendritic cells and/or directly with T cells to stimulate autologous T cells with primed dendritic cells (DC). (Day 1: (AM) IgG+ (DC:tumor=1:1); (PM) transfer to new plate; add LPS; TC-treated) (Day 2: TC-treated; (T cell:DC=5:1)+IL7) (Day 4, add IL6 and IL15) (Day 6, not TC-treated, transfer to plate with αCD3/αCD28/α4-1BB) (Day 7, TC-treated, transfer to a new plate, add IL2) (Day 8-9, feed T cells as needed with IL15) (Day 10, harvest T cells, ELISpot, cytotoxicity assay, phenotype)

### Example 2

In an exemplary method, PBNP-PTT heating of cancer cells was performed in a laser power-dependent manner. In brief, five million cells—tumor or normal, were suspended in 500 μL media with 0.15 mg/mL PBNPs. The samples were then illuminated by the NIR laser for 10 minutes at varied powers (e.g. 0.75, 1.0, 1.5 or 2 W). Temperatures were measured using a thermal camera (FLIR, Arlington, VA, USA), and recorded every minute for 10 minutes.

The extent of thermal damage to tissue depends on tissue sensitivity, temperature and exposure time. To clarify the threshold for thermal damage PBNPs may cause when used as a photothermal therapy agent, the thermal dose (CEM43° C.) was calculated using thermal histories from the above in vitro data. Specifically the time-temperature history is converted to an equivalent number of minutes of heating at 43° C., using the following formula:

$$CEM 43 ° C . = {\sum }_{i = 1}^{n} {t}_{i} \cdot {R}^{( 43 - {T}_{i} )} ,$$

where the equivalent number of minutes of heating at 43° C. is a model to calculate a thermal isoeffect dose.

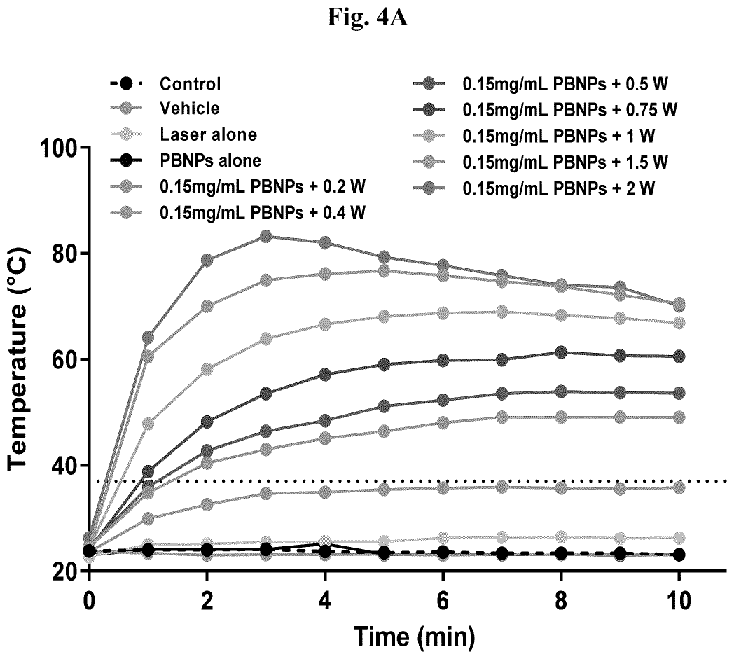

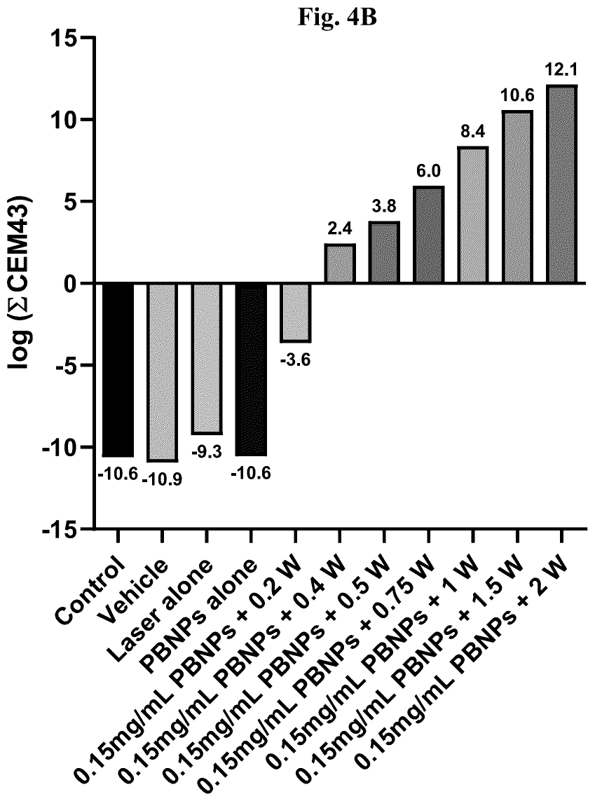

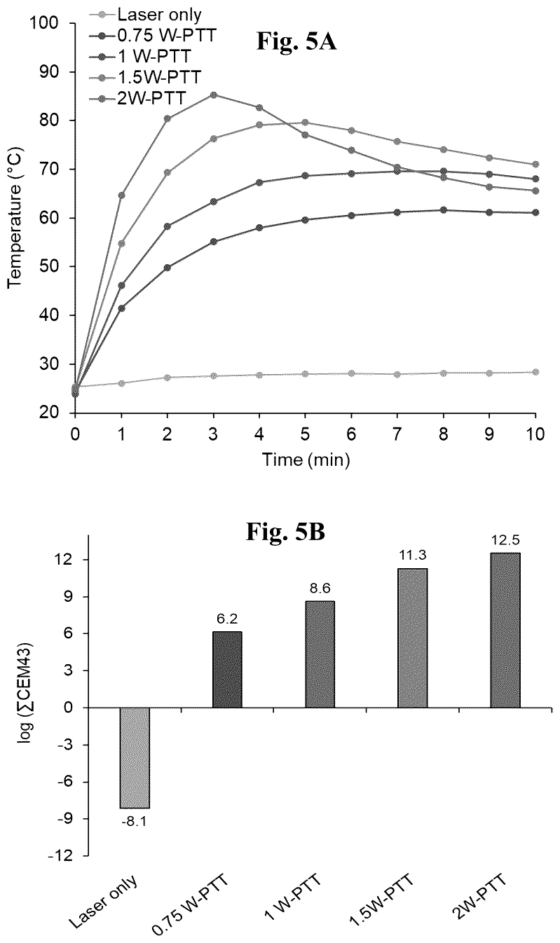

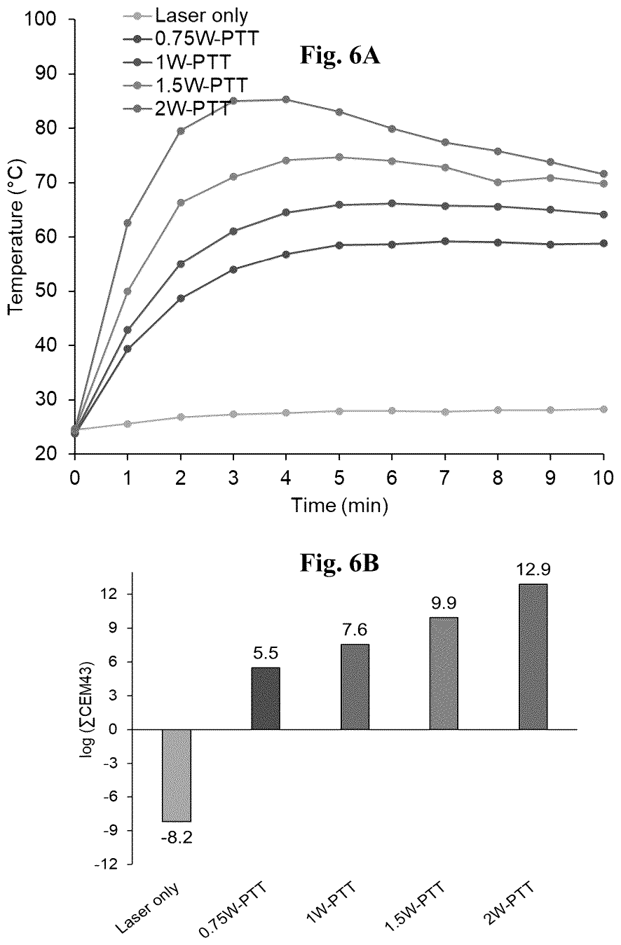

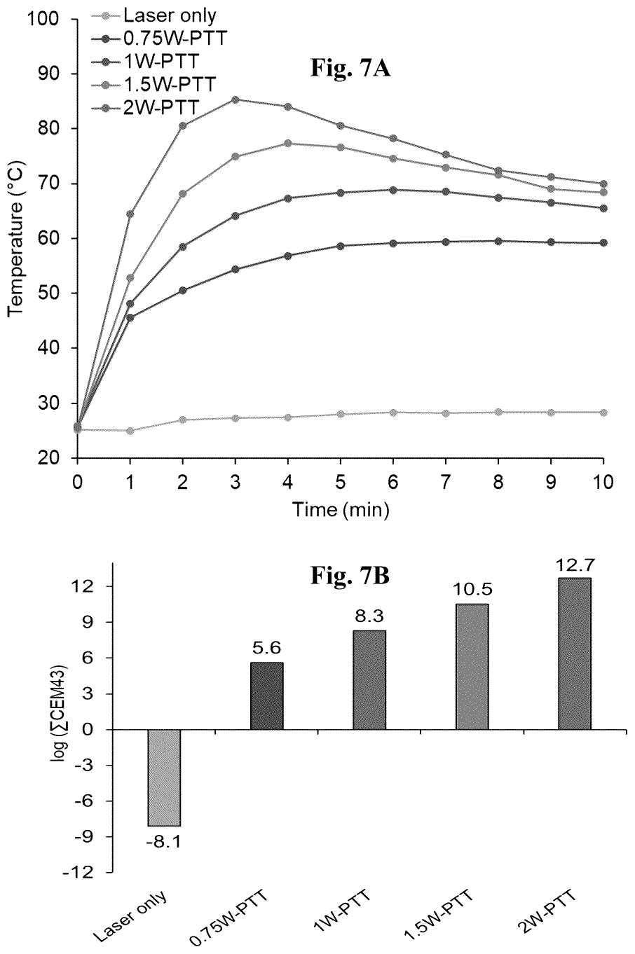

Cancer cells tested herein using the above methods included glioblastoma (GBM) cells (U87; SNB-19), neuroblastoma cells (SH-SY5Y), and breast cancer cells (MDA-MB-231). FIGS. 4A and 4B show that PBNP-PTT heated U87 glioblastoma (GBM) cells in laser power-dependent manner. FIGS. 5A and 5B show that PBNP-PTT heated SNB-19 glioblastoma (GBM) cells in laser power-dependent manner. FIGS. 6A and 6B show that PBNP-PTT heated SH-SY5Y (neuroblastoma) cells in laser power-dependent manner. FIGS. 7A and 7B show that PBNP-PTT heated MDA-MB-231 (breast cancer) cells in laser power-dependent manner.

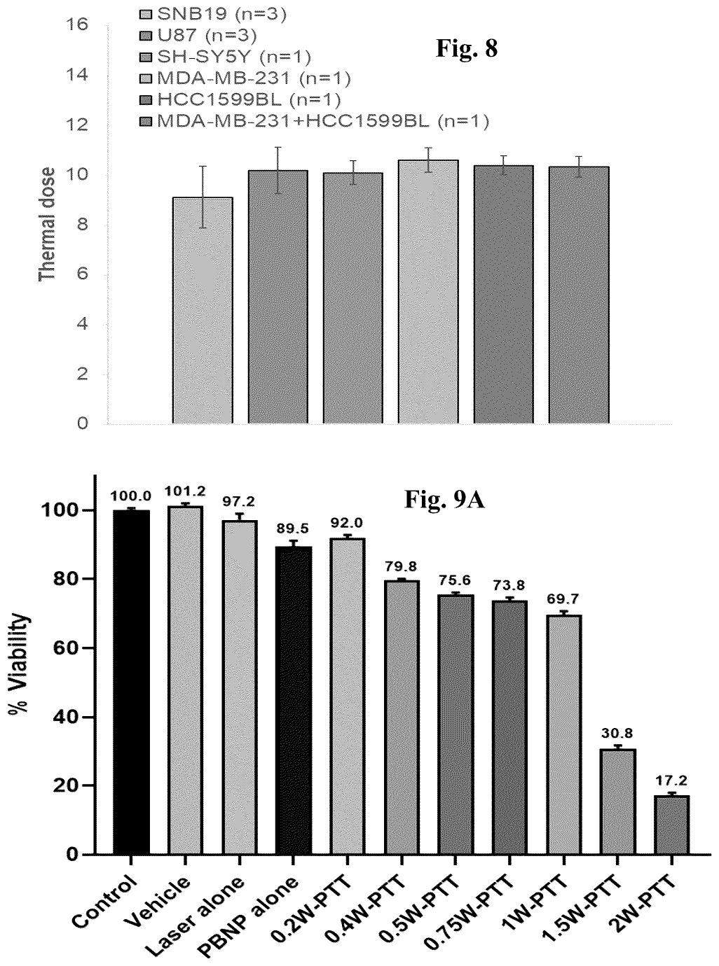

Thermal dose (CEM43° C.) was calculated for GBM cells (U87; SNB-19), neuroblastoma cells (SH-SY5Y), breast cancer cells (MDA-MB-231), and HCC1599 BL cells—a B lymphoblastoid cell line derived from a female with primary ductal carcinoma of the breast. FIG. 8 shows that PBNP-PTT consistently heated tumor cell lines to a thermal dose ˜10 log (CEM43).

### Example 3

In another exemplary method, the effect of increasing thermal dose (i.e., higher laser power) on immunogenicity was assessed in cancer cells by measuring expression of surface markers associated with immunogenic cell death (ICD): ATP, HMGB1, and calreticulin. In brief, cancer cells were suspended in 500 μL media with 0.15 mg/mL PBNPs and illuminated by the NIR laser for 10 minutes at varied powers (e.g. 0.75, 1.0, 1.5 or 2 W). Cells were then collected by centrifugation (800 g, 5 minutes), washed twice in fluorescence-activated cell sorting (FACS) buffer, and stained for 20 minutes at room temperature (23° C.±3° C.) using monoclonal antibodies against the surface activation markers ATP, HMGB1, and calreticulin. After staining, cells were washed twice and resuspended in FACS buffer containing DAPI (4′,6-diamidino-2-phenylindole) for analysis by flow cytometry. All flow cytometry data was conducted by using FlowJo software.

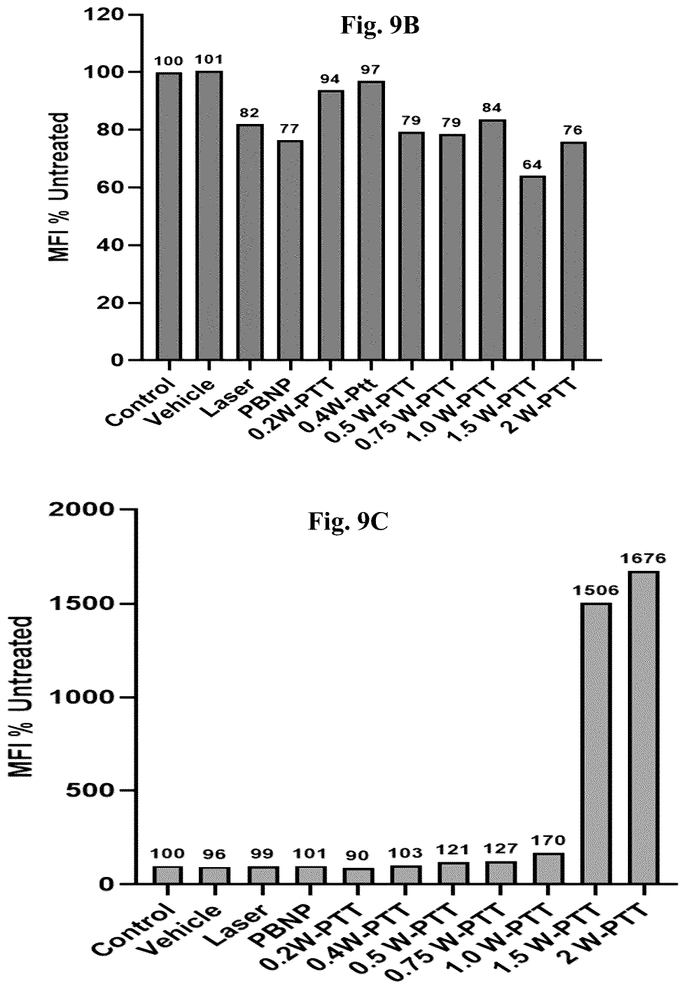

As shown in FIGS. 9A-9C, U87 cells expressed ATP, HMGB1, and calreticulin in amounts comparable to control cells when subjected to 0.2 W, 0.4 W, 0.5 W, 0.75 W, and 1 W PPT. However, U87 cells treated with either 1.5 W or 2 W showed decreased ATP expression (FIG. 9A), decreased HMGB1 expression (FIG. 9B), and increased calreticulin expression (FIG. 9C).

In addition to measuring cell surface markers, cell viability and ATP release were also determined for cancer cells subjected to PBNP-PTT. To calculate intracellular ATP, cells were harvested 24 hours after in vitro PBNP-PTT, washed with 1× PBS, and mixed with the ATP reagent from the CellTiter-Glo Luminescent Cell Viability Assay. Luminescence was then measured using a SpectraMax i3X microplate reader whereupon luminescence was correlated to intracellular ATP as a measure of ATP released from the cells. Also after the 24-hour incubation period, cell viability was assessed using a luminescent cell viability assay.

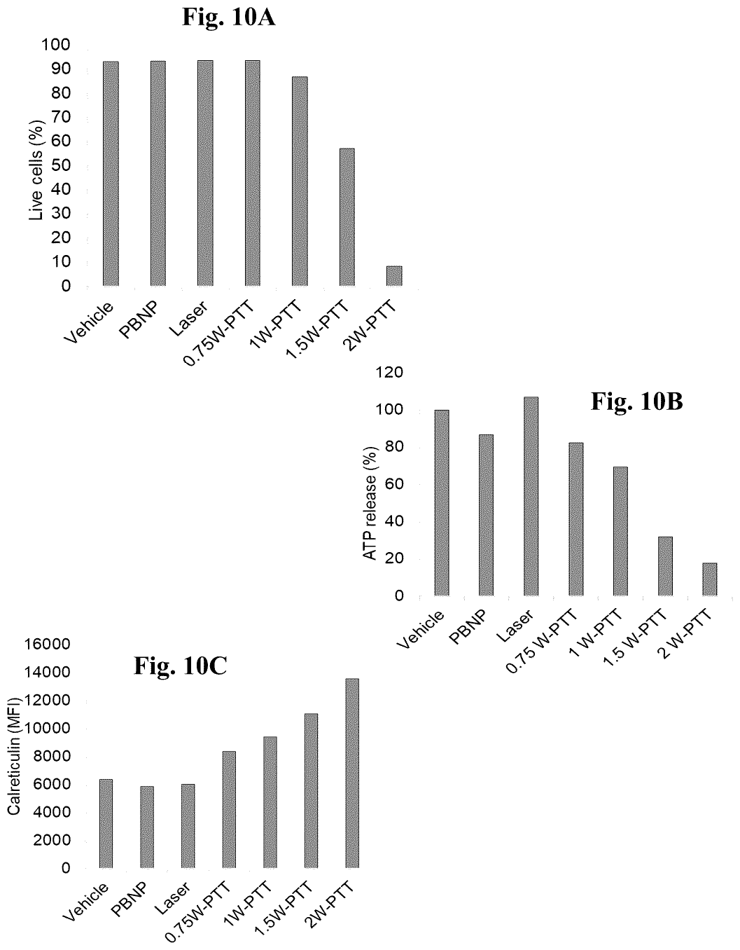

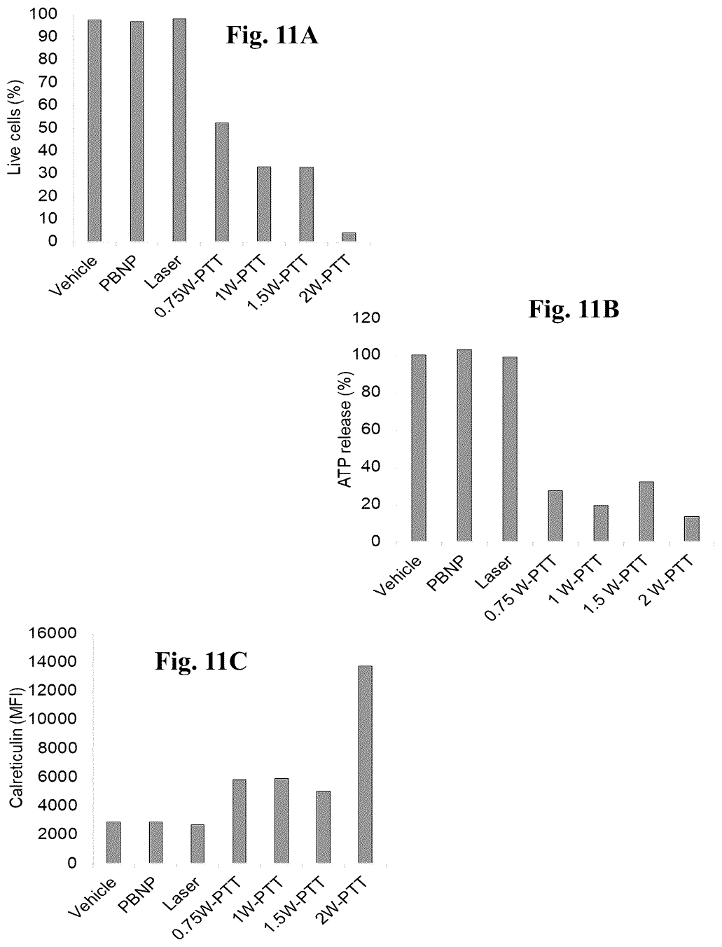

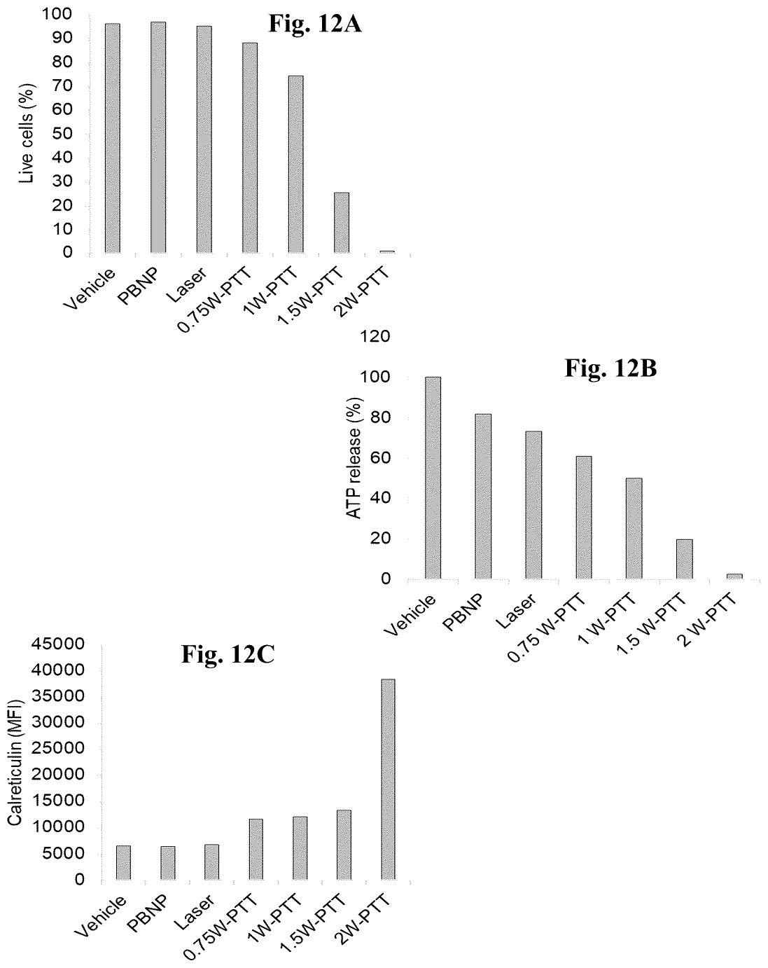

PBNP-PTT increased immunogenicity of SNB-19 cells as evidenced by a decrease in the number of live cells (FIG. 10A), a decrease in intracellular ATP (FIG. 10B), and an increase in calreticulin cell expression (FIG. 10C). PBNP-PTT increased immunogenicity of SH-SY5Y cells as evidenced by a decrease in the number of live cells (FIG. 11A), a decrease in intracellular ATP (FIG. 11B), and an increase in calreticulin cell expression (FIG. 11C). PBNP-PTT increased immunogenicity of MDA-MB-231 cells as evidenced by a decrease in the number of live cells (FIG. 12A), a decrease in intracellular ATP (FIG. 12B), and an increase in calreticulin cell expression (FIG. 12C).

### Example 4

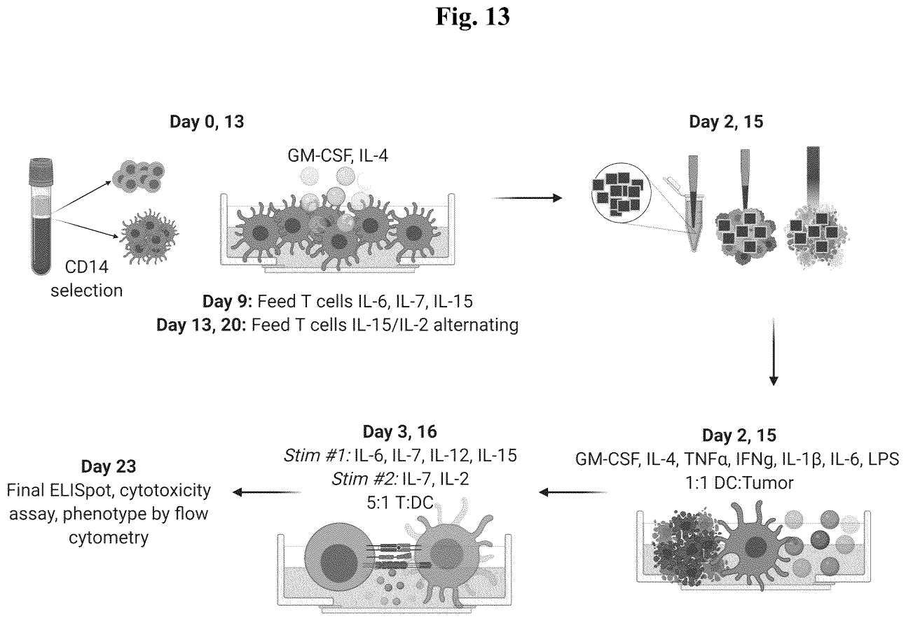

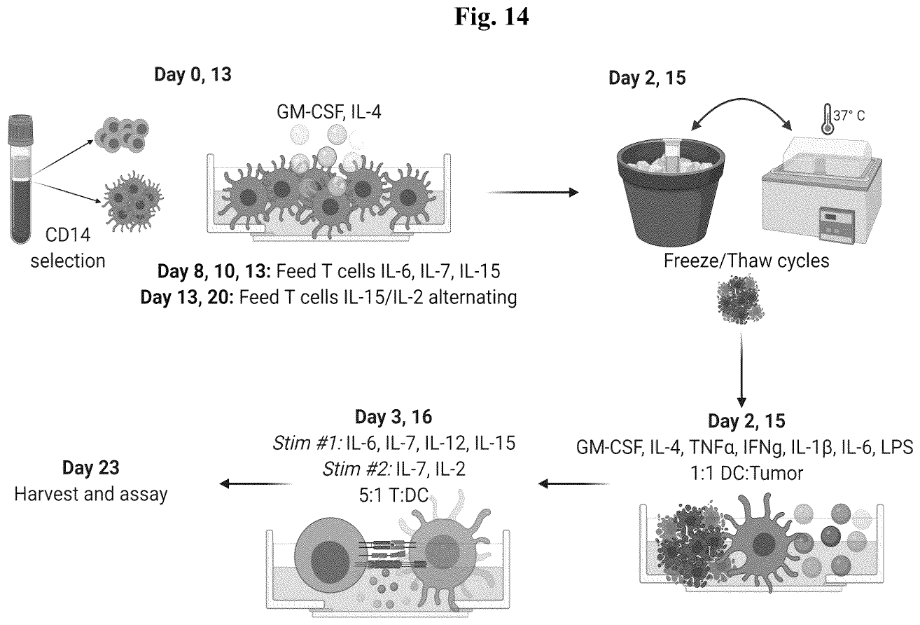

In another exemplary method, T cells were manufactured. In brief, T cell lines were generated from PBMCs that were HLA-matched to U87, SNB-19, SH-SY5Y, MDA-MB-231 and HCC1599 BL cells using monocyte-derived dendritic cells (DCs). Monocytes were isolated by CD14 isolation MACS beads kit. The isolated cells were cultured in DC media in the presence of IL-4 (1000 U/mL) and granulocyte-macrophage colony-stimulating factor GM-CSF (800 U/mL) cytokines. One day prior to stimulation, DCs for each donor were pulsed with cells subjected to PBNP-PTT at 1.5 W laser power (FIG. 13) or cell lysate generated using multiple freeze-thaw cycles using a dry ice-ethanol mixture and a water bath maintained at 37° C. (FIG. 14). DCs were then matured with GM-CSF, TNF-a, IL-1b, IL4, IL-6, GM-CSF, IFN-γ, IL-4 and lipopolysaccharide (LPS) overnight. Following maturation, CD14 negative PBMCs were thawed and stimulated with harvested DCs at a 1:5 (DC:T cell) ratio in CTL media supplemented with IL-6, IL-7, IL-12 and IL-15 (R&D systems). Cells were incubated at 37° C. incubator and were expanded and fed with fresh cytokines as necessary. Subsequent stimulation was performed in a similar way. On day 23-24, cells were harvested for the functional studies.

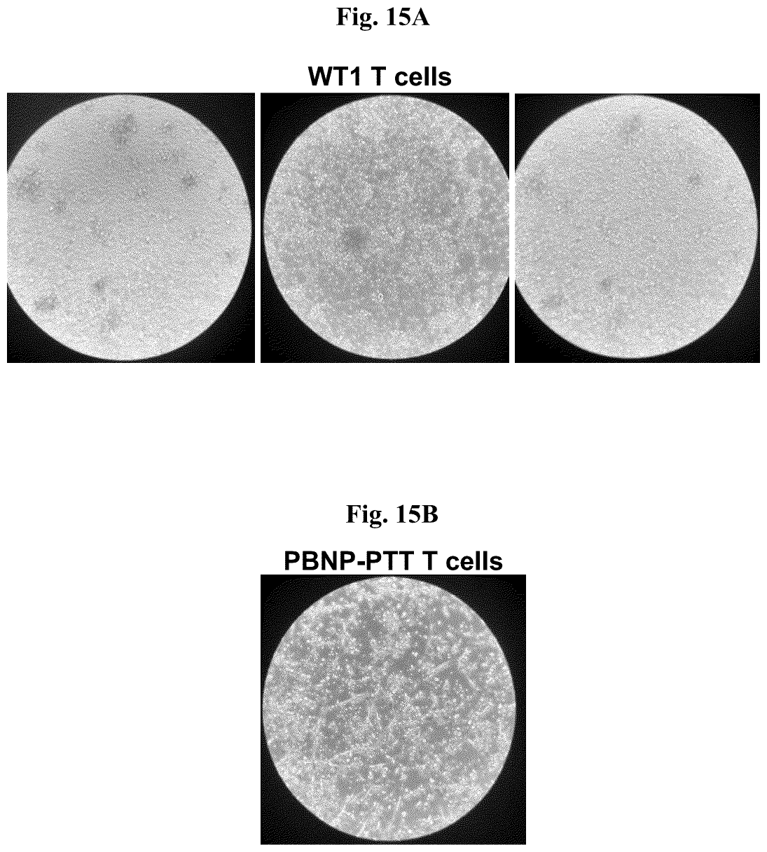

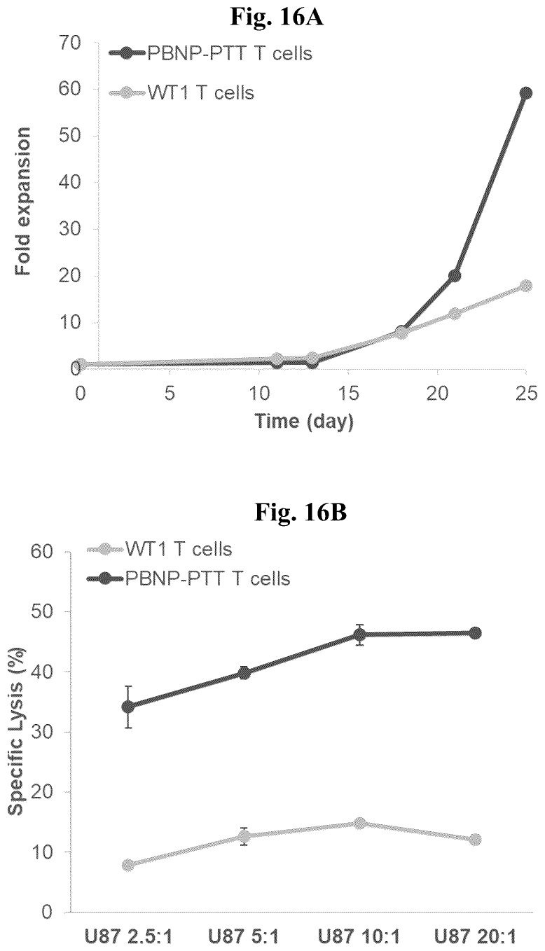

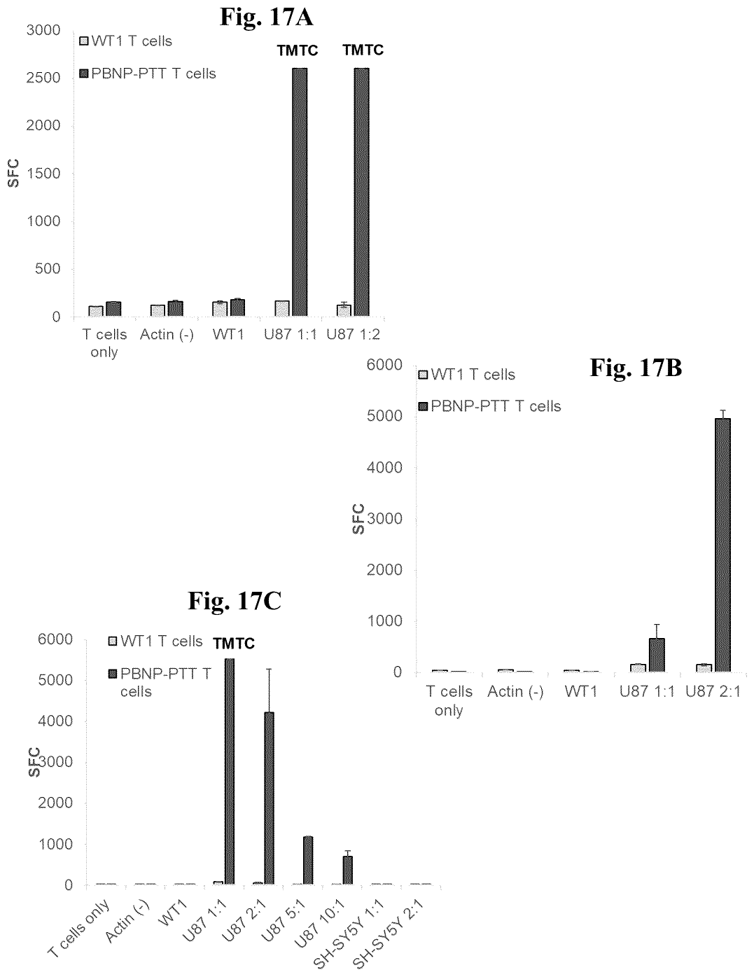

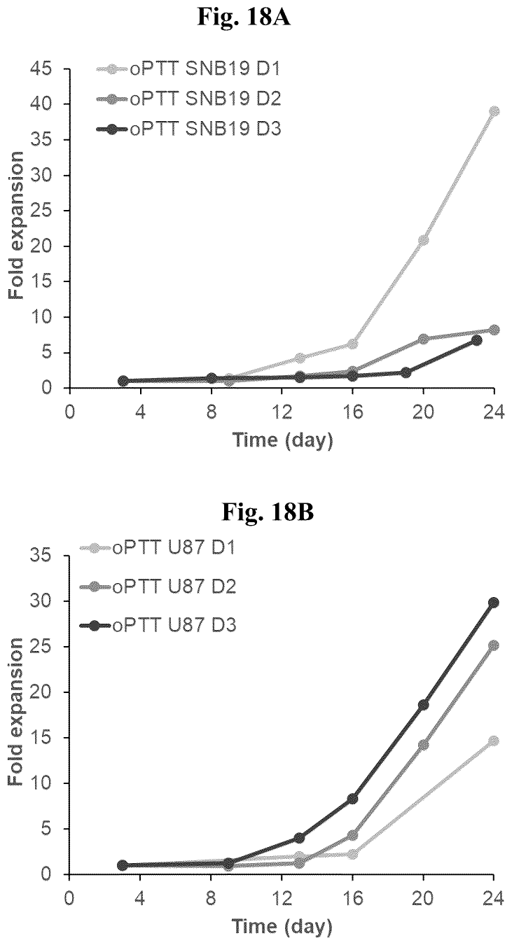

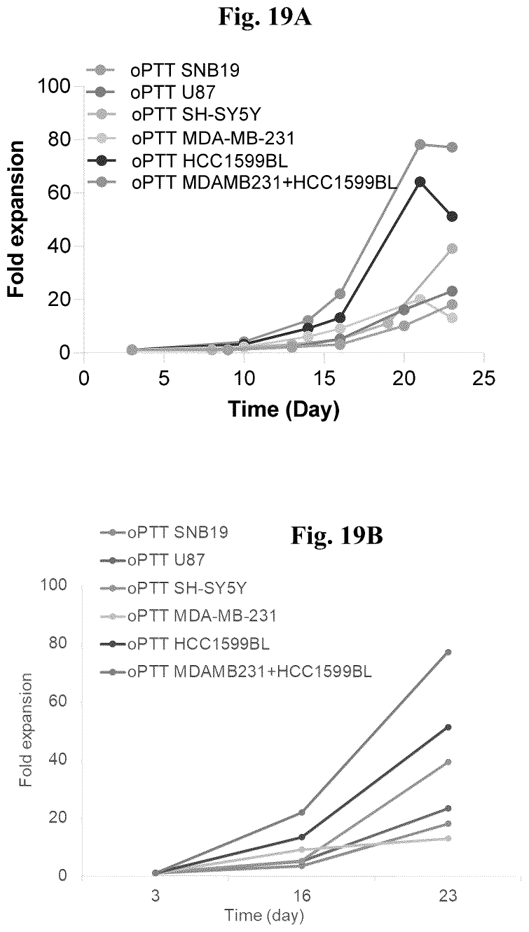

FIGS. 15A and 15B show U87-specific T cells that were not subjected to PBNP-PTT (FIG. 15A) and cells that were subjected to PBNT-PTT (FIG. 15B) at day 11 for 7 days post-Stim no. 1. FIGS. 16A and 16B shows the manufacture of U87-specific T cells, which was enabled using PBNP-PTT. FIGS. 17A-17C shows the manufacture of U87-specific T cells, which was enabled using PBNP-PTT, regarding post-Stim 1 (FIG. 17A), post-Stim 2 (FIG. 17B), and post-Stim 3 (FIG. 17C). PBNP-PTT-mediated development expanded T cells˜7-40-fold over 24 days in SNB19-specific T cells (FIG. 18A) and U87-specific T cells (FIG. 18B). FIGS. 19A and 19B shows that PBNP-PTT-mediated development expands T cells˜10-80 fold over 23 days when using the cancer cell lines SNB19, U87, SH-SY5Y, MDA-MB-231, HCC1599BL and MDA-MB-231+HCC1599BL.

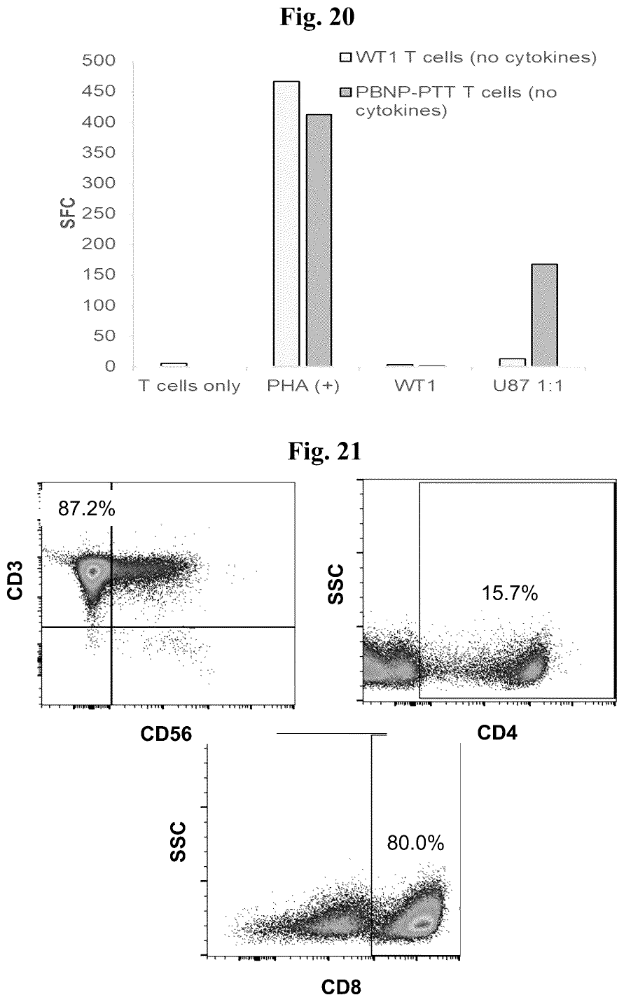

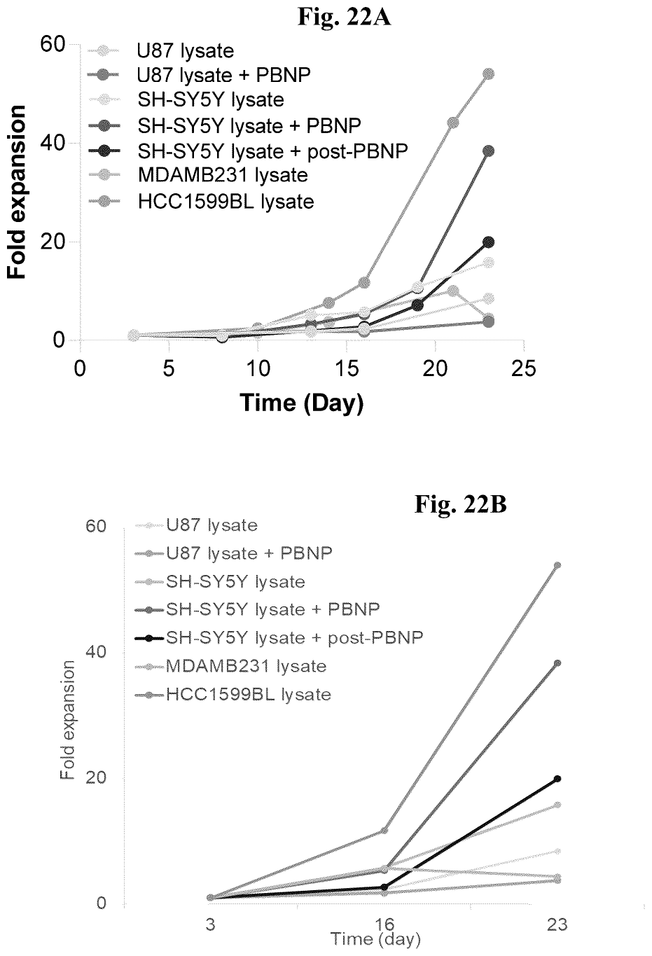

FIG. 20 shows the generation of U87-specific T cells in the absence of cytokines, which was enabled using PBNP-PTT. FIGS. 22A and 22B shows that lysate-mediated T cells expansion was lower than PBNP-PTT T cells.

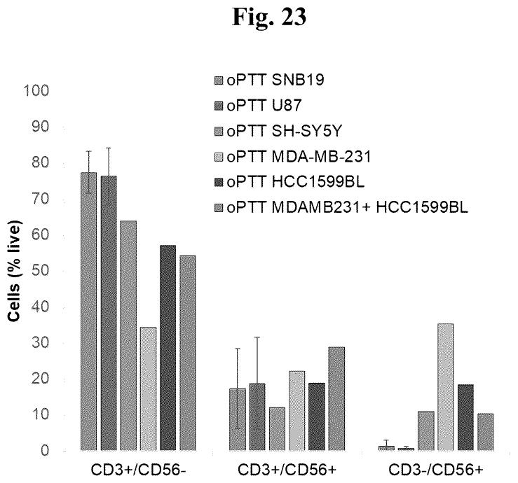

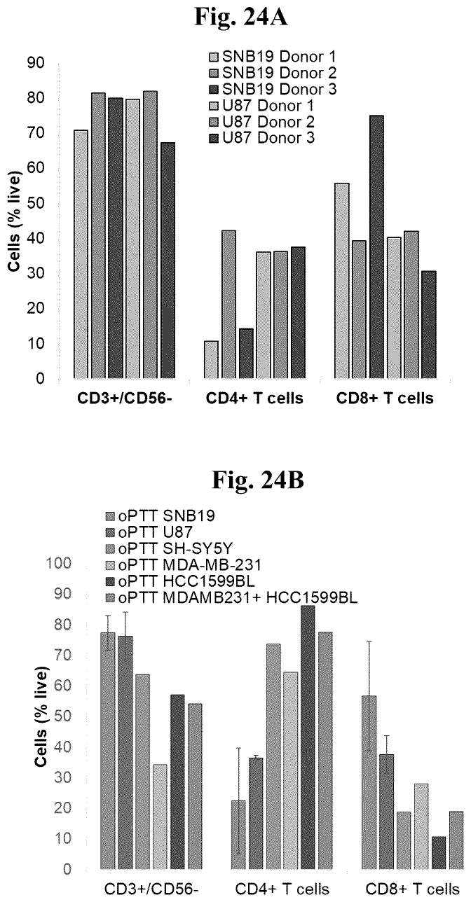

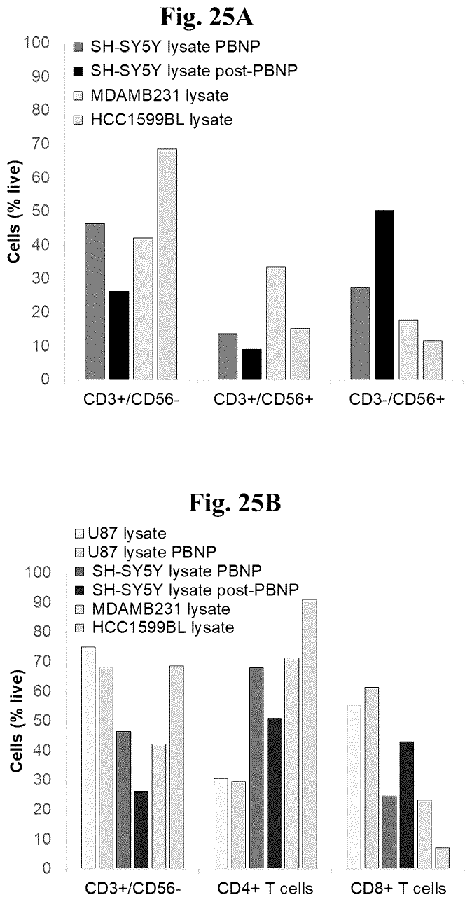

To assess the T cell population that resulted from PBNP-PTT-mediated cell expansion, cells were harvested and subjected to FACS analysis using methods disclosed herein. FIG. 21 shows that PBNP-PTT-mediated cell expansion resulted in the manufacture of primarily CD8+ U87-specific T cells. FIG. 23 shows that cells expanded via PBNP-PTT consists majority of T cell population. FIGS. 24A and 24B show that T cells expanded via PBNP-PTT were a mixed of CD4+ and CD8+ population. FIGS. 25A and 25B show that cells expanded via lysate had higher CD4+ T cell populations.

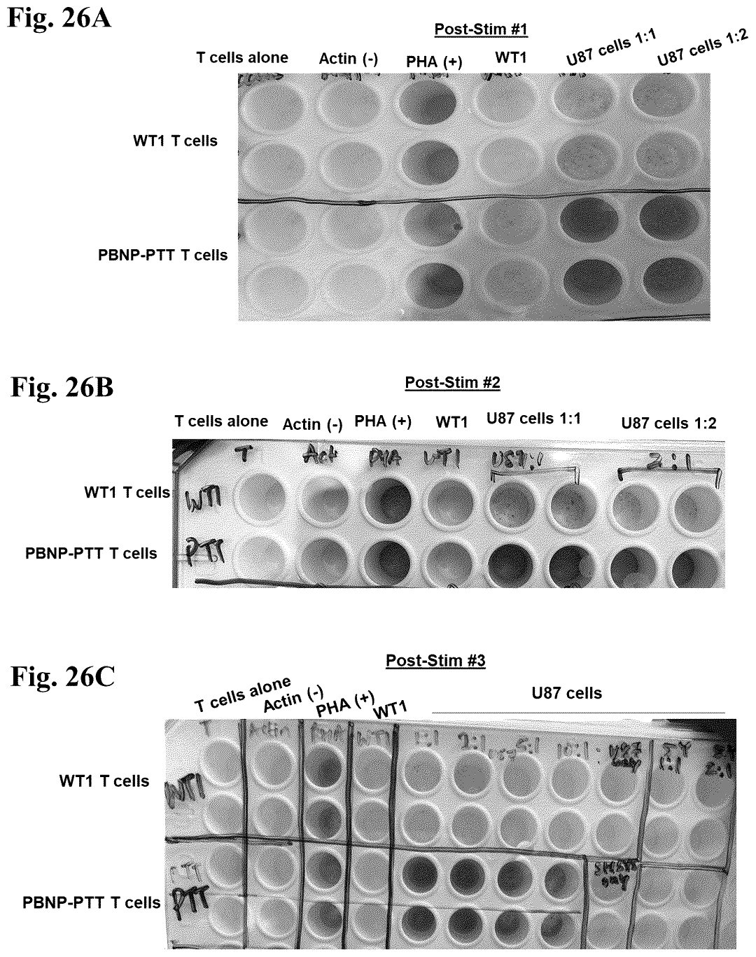

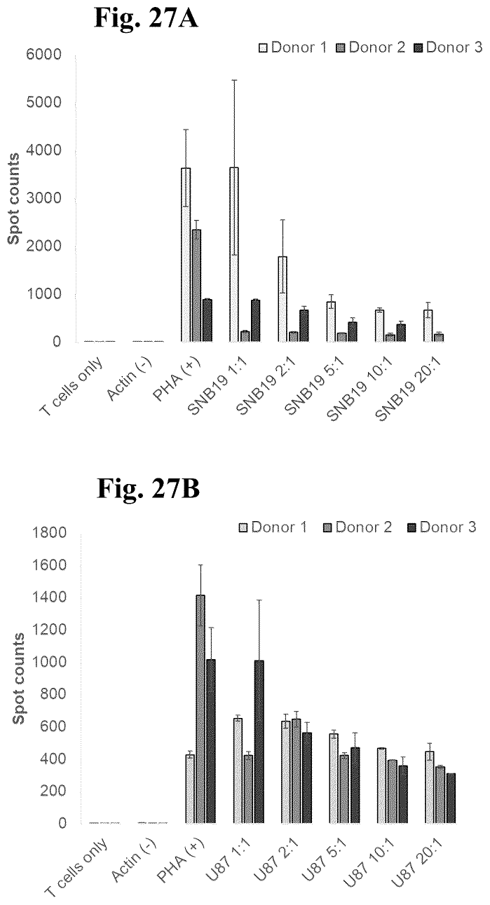

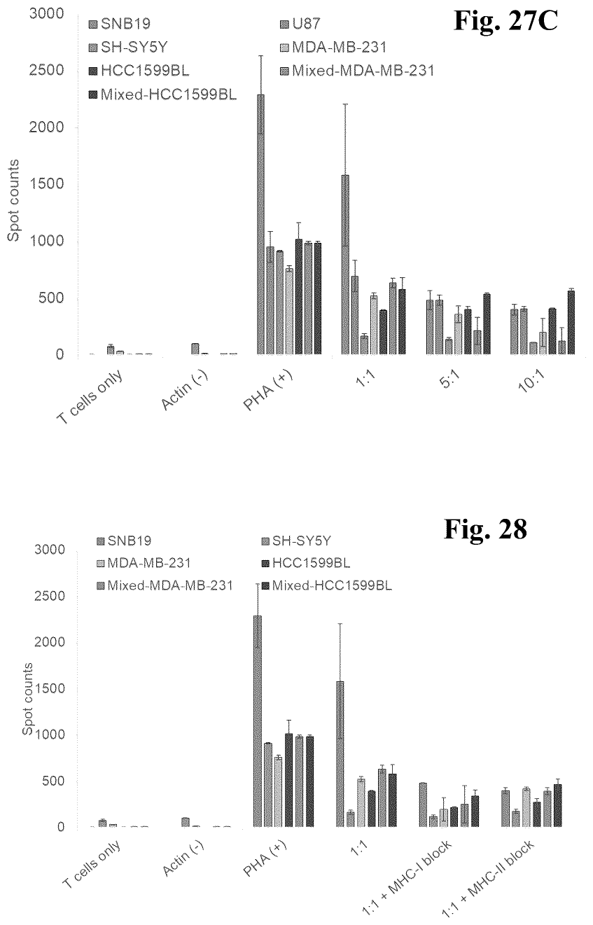

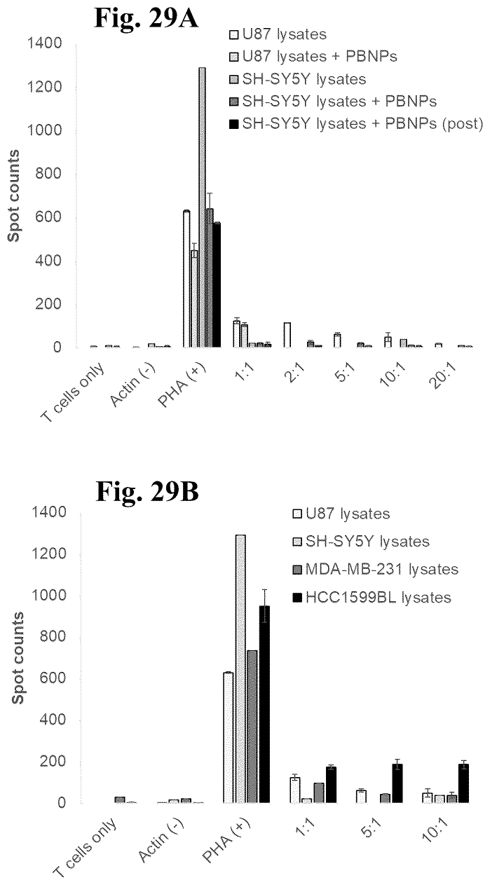

T cell specificity was determined by IFN-γ ELISPOT. ELISPOT plate was coated with IFN-γ capture antibody. Following day, target cells were collected and used either directly or incubated with anti-HLA-ABC or anti-HLA-DR/DP/DQ antibodies for 1 hour at 37° C. as blocking antibodies. T cells were either plated alone or in presence of the target cells at different effector:target (E:T) ratio. Actin was used as a negative control and PHA was used as a positive control. Cells were incubated at 37° C. and 24 hours later plate was developed. FIGS. 26A-26C show representative images of ELISPOT plates developed for U87-specific T cells post stimulation #1 (FIG. 26A), #2 (FIG. 26B), and #3 (FIG. 26C). Number of IFN-γ spot-forming units were calculated and quantitated. FIGS. 27A-27C show that T cells developed via PBNP-PTT-mediated expansion secreted IFN-γ in response to target cells. FIG. 28 shows that T cells developed via PBNP-PTT-mediated expansion secrete IFN-γ in response to target cells via. MHC-I signaling. FIGS. 29A and 29B show that T cells developed via lysate-mediated expansion secreted lower IFN-γ in response to target cells.

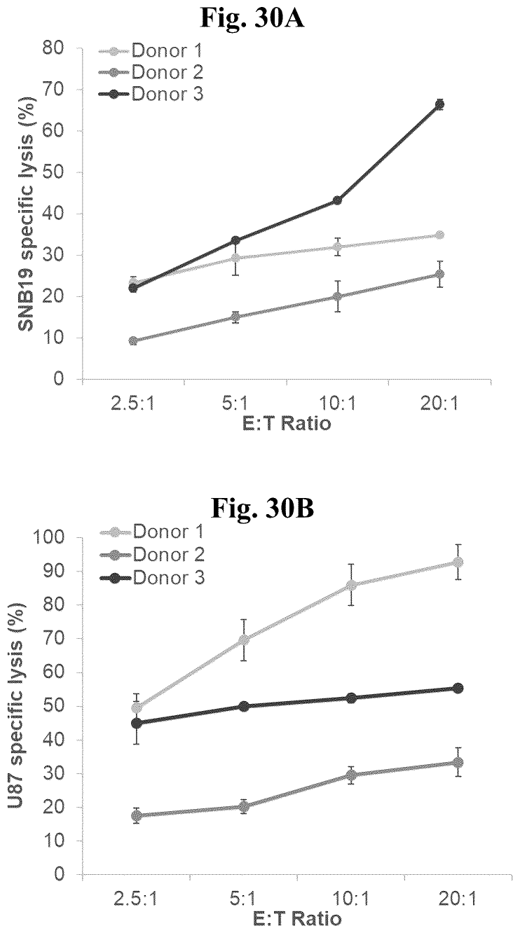

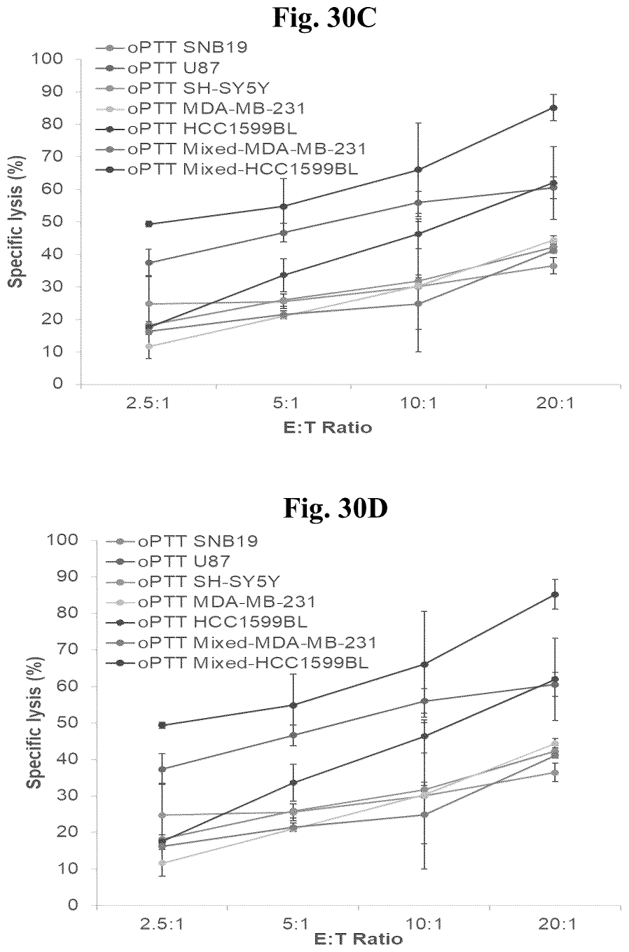

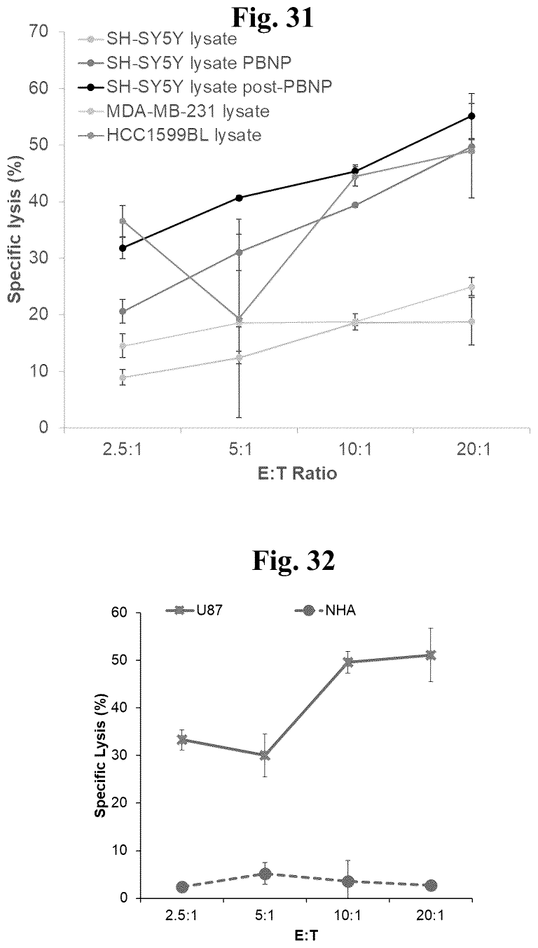

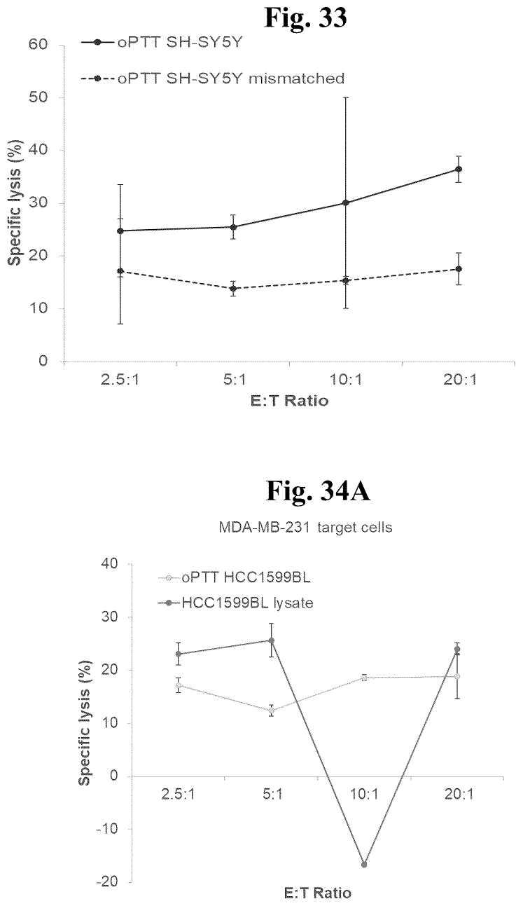

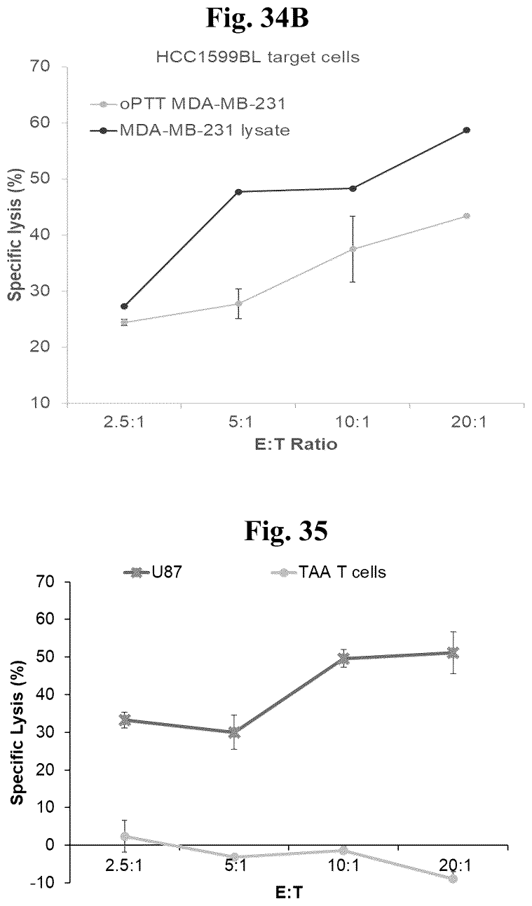

Cytotoxicity against target cancer cells was assessed in T cells developed via PBNP-PTT-mediated expansion. In brief, target cells were stained with Calcein-AM for 30 min at 37° C. For blocking antibodies, target cells were incubated with either anti-HLA-ABC or anti-HLA-DR/DP/DQ antibodies for 1 hour at 37° C. Expanded T cells were re-suspended at different concentrations and were mixed with target cells to generate different effector:target (E:T) ratio. Spontaneous and total release were set up using cells with either media alone or using 2% Triton X-100 respectively. Plate was incubated for 4 hours and supernatant was used to calculate the fluorescence reading. Specific analysis was calculated using (Sample measurement−Spontaneous release)/(Total release−Spontaneous release)*100. FIGS. 30A-30D show that T cells developed via PBNP-PTT-mediated expansion were cytotoxic against target cells. FIG. 31 shows that T cells developed via lysate-mediated expansion were cytotoxic against target cells. FIG. 32 shows that PBNP-PTT T cells were not cytotoxic to unmatched normal human astrocytes (NHAs). FIG. 33 shows that SH-SY5Y PBNP-PTT T cells generated from a mismatched donor were not cytotoxic to its target cells. FIGS. 34A and 34B shows T cells generated via PBNP-PTT had lower cytotoxicity to off-target cells as compared to lysate developed T cells. FIG. 35 shows T cells generated via PBNP-PTT were more cytotoxic toward U87 cells than tumor-associated antigens (TAA T) cells.

Exemplary methods disclosed herein demonstrated that: 1) PBNP-PTT induced immunogenicity in the tested cell lines—U87, SNB-19, SH-SY5Y and MDA-MB-231; 2) PBNP-PTT and lysate-mediated T cell expansion was cell line and donor dependent; 3) Cells expanded via a PBNP-PTT or a lysate method had a mixed population of CD4+ and CD8+ T cells and the percentage of its population varied with tumor type; 4) T cells developed via PBNP-PTT had higher IFN-γ release cells in response to its target cells as compared to lysate generated T cells and TAA T cells; 5) SNB-19 and MDA-MB-231 specific T cells secreted IFN-γ in response to target cells via MHC-I signaling; 6) T cells developed via PBNP-PTT and lysate-mediated were cytotoxic against target cells; 7) SH-SY5Y T cells developed via tumor lysate-mediated expansion were more cytotoxic against target NB cells in the presence of PBNPs; 8) U87 T cells and SH-SY5Y T cells from a mismatched donor developed via PBNP-PTT were not cytotoxic to unmatched normal human astrocytes (NHAs) and to SH-SY5Y target cells respectively; 9) T cells generated via PBNP-PTT had lower cytotoxicity to off-target cells (HLA matched) as compared to the lysate developed T cells.

All the COMPOSITIONS and METHODS disclosed and claimed herein may be made and executed without undue experimentation in light of the present disclosure. While the COMPOSITIONS and METHODS have been described in terms of preferred embodiments, it will be apparent to those of skill in the art that variation may be applied to the COMPOSITIONS and METHODS and in the steps or in the sequence of steps of the METHODS described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to he within the spirit, scope and concept of the invention as defined by the appended claims.

1. A method of preparing ex vivo expanded immune cells, the method comprising:
obtaining an initial immune cell population;
isolating monocyte-derived dendritic cells (DCs) and T cells from the initial immune cell population;
obtaining at least one target cancer cell;
exposing the at least one target cancer cell to at least one Prussian blue nanoparticle (PBNP) and subjecting the at least one target cancer cell and the at least one PBNP to photothermal therapy (PTT);
co-culturing the isolated monocyte-derived DCs with the at least one target cancer cell subjected to PTT;
expanding the co-culture in a medium comprising the isolated T cells; and
harvesting from the co-culture expanded immune cells specific to the at least one target cancer cell.

2. The method of claim 1, wherein the initial immune cell population is obtained from peripheral blood mononuclear cells (PBMCs), a leukapheresis sample, tumor-infiltrated lymphocytes, tissue-infiltrated lymphocytes, lymph nodes, a thymus, secondary lymphoid organs, or any combination thereof.

3. The method of claim 1, wherein the at least one PBNP comprises a Prussian blue material represented by general formula (I):
AₓB_{y}M₄[M′(CN)₆]_{z}·nH₂O (I)
wherein:
A represents at least one of VO₂, Ca, V, Cr, Mn, Fe, Co, Ni, Cu, Zn, Ga, Sr, Zr, Nb, Li, Na, K, Rb, Cs, Fr, Tl, Mo, Ru, Rh, Pd, Ag, Cd, In, Lu, Ba, Hf, Ta, W, Os, Pt, Hg, La, Eu, Gd, Tb, Dy and Ho, in any oxidation state and any combination thereof;
B represents at least one of VO₂, Ca, V, Cr, Mn, Fe, Co, Ni, Cu, Zn, Ga, Sr, Zr, Nb, Li, Na, K, Rb, Cs, Fr, Tl, Mo, Ru, Rh, Pd, Ag, Cd, In, Lu, Ba, Hf, Ta, W, Os, Pt, Hg, La, Eu, Gd, Tb, Dy and Ho, in any oxidation state and any combination thereof;
M represents at least one of VO₂, Ca, V, Cr, Mn, Fe, Co, Ni, Cu, Zn, Ga, Sr, Zr, Nb, Li, Na, K, Rb, Cs, Fr, Tl, Mo, Ru, Rh, Pd, Ag, Cd, In, Lu, Ba, Hf, Ta, W, Os, Pt, Hg, La, Eu, Gd, Tb, Dy and Ho, in any oxidation state and any combination thereof;
M′ represents at least one of VO₂, Ca, V, Cr, Mn, Fe, Co, Ni, Cu, Zn, Ga, Sr, Zr, Nb, Li, Na, K, Rb, Cs, Fr, Tl, Mo, Ru, Rh, Pd, Ag, Cd, In, Lu, Ba, Hf, Ta, W, Os, Pt, Hg, La, Eu, Gd, Tb, Dy and Ho, in any oxidation state and any combination thereof;
x is from 0.1 to about 1;
y is from 0.1 to about 1;
z is from 0.1 to about 4; and
n is from 0.1 to about 24.

4. The method of claim 1, wherein the PTT comprises the use of a device that emits electromagnetic radiation with a wavelength that irradiates the at least one PBNP exposed to the at least one target cancer cell.

5. The method of claim 1, wherein the co-culturing of the isolated monocyte-derived DCs with the at least one target cancer cell subjected to PTT occurs in the presence of GM-CSF, TNF-a, IL-1b, IL4, IL-6, GM-CSF, IFN-γ, IL-4, lipopolysaccharide, or any combination thereof.

6. The method of claim 1, wherein the isolated T cells are stimulated with DCs harvested from the co-culture.

7. The method of claim 6, wherein the isolated T cells are stimulated with DCs harvested from the co-culture in the presence of IL-6, IL-7, IL-12, IL-15, or any combination thereof.

8. The method of claim 1, wherein the ex vivo expanded immune cells specific to the at least one target cancer cell have at least one marker of T cell activation, and wherein the at least one marker of T cell activation comprises CD45RO, CD137, CD25, CD279, CD179, CD62L, HLA-DR, CD69, CD223 (LAG3), CD134 (0X40), CD183 (CXCR3), CD27 (IL-7Ra), CD366 (TIM3), CD80, CD152 (CTLA-4), CD28, CD278 (ICOS), CD154 (CD40L), or any combination thereof.

9. The method of claim 1, wherein the initial immune cell population is matched to the at least one target cancer cell on at least 1 human leukocyte antigen (HLA).

10. The method of claim 1, wherein the initial immune cell population and the at least one target cancer cell are obtained from a subject having cancer.

11. The method of claim 1, wherein the ex vivo expanded immune cells comprise T cells specific for the subject's cancer.

12. The method of claim 11, wherein the T cells specific for the subject's cancer comprise CD8+ T cells, CD4+ T cells, or any combination thereof.

13. An immunotherapy composition, comprising:
the ex vivo expanded immune cells specific for at least one target cancer cell harvested in claim 1.

14. A method of treating a subject in need thereof, the method comprising:
administering to a subject having cancer the immunotherapy composition of claim 13.

15. The method of claim 14, wherein the initial immune cell population and the at least one target cancer cell are obtained from the subject having cancer.

16. The method of claim 15, wherein administering to the subject the immunotherapy composition comprises infusion.

17. The method of claim 14, wherein the cancer comprises breast cancer, colorectal cancer, head and neck cancer, kidney cancer, lung cancer, ovarian cancer, pancreatic cancer, prostate cancer, brain cancer, adenoid cystic carcinoma, anaplastic astrocytoma, anaplastic ependymoma, anaplastic oligodendroglioma, brainstem glioma, diffuse astrocytoma, diffuse intrinsic pontine glioma (DIPG), ganglioglioma, medulloblastoma, pilocytic astrocytoma, cholangiocarcinoma, chronic atypical myelogenous leukemia, endometrial carcinoma, esophageal cancer, Ewing sarcoma, gastrointestinal stromal tumor (GIST), leptomeningeal carcinomatosis, multiple myeloma, myelodysplastic syndrome, neuroendocrine carcinoma, Non-Hodgkin's lymphoma, pleomorphic sarcoma, primitive neuroectodermal tumor (PNET), refractory anemia, salivary gland carcinoma, skin cancer, stomach cancer, thyroid cancer, urothelial cancer, or any combination thereof.

18. The method of claim 14, further comprising administering to the subject one or more chemotherapeutic agents, monoclonal antibody therapies, small molecules, or any combination thereof.

19. The method of claim 18, wherein the one or more monoclonal antibody therapies comprise adotrastuzumab, trastuzumab, pertuzumab, cetuximab, panitumumab, necitumumab, ramucirumab, bevacizumab, rituximab, ofatumumab, ibritumomab, tositumomab, obinutuzumab, inotuzumab, alemtuzumab, gemtuzumab, brentuximab, blinatumomab, daratumumab, ipilimumab, nivolumab, atezolizumab, avelumab, cemiplimab, pembrolizumab, durvalumab, denosumab, dinutuximab, olaratumab, elotuzumab, or any combination thereof.

18. The method of claim 18, wherein the one or more small molecules comprise imatinib, dasatinib, nilotinib, bosutinib, regorafenib, ponatinib, sunitinib, sorafenib, erdafitinib, lenvatinib, pazopanib, afatinib, gefitinib, osimertinib, vandetanib, erlotinib, lapatinib, dacomitinib, neratinib, ribociclib, abemaciclib, palbociclib, cabozantinib, crizotinib, axitinib, alectinib, vemurafenib, encorafenib, dabrafenib, olaparib, rucaparib, talazoparib, niraparib, larotrectinib, entrectinib, lorlatinib, ibrutinib, cobimetinib, binimetinib, trametinib, brigatinib, cgilteritinib, ceritinib, ivosidenib, carfilzomib, marizomib, alpelisib, duvelisib, copanlisib, or any combination thereof.
